(19) United States
(12) Patent Application Publication
(10) Pub. No.: US 2009/0047340 A1
(43) Pub. Date: Feb. 19, 2009

(54) LIPOSOMAL REDUCED GLUTATHIONE AND 1-ARGININE, INCLUDING WITH OTHER INGREDIENT(S), CAPABLE OF MULTIPATH ADMINISTRATION FOR REVERSAL AND PREVENTION OF OBESITY AND FOR MITOCHONDRIAL BIOGENESIS

(76) Inventor: F. Timothy Guilford, Palo Alto, CA (US)

Correspondence Address: BROOKE SCHUMM III;Daneker, McIntire, Schumm, Prince, Goldstein et al, ONE NORTH CHARLES STREET, SUITE 2450, BALTIMORE, MD, 21201, US

(21) Appl. No.: 12/281,066

(22) PCT Filed: Mar. 29, 2007
(86) PCT No.: PCT/US07/65552

§ 371 Date: Aug. 28, 2008

Related U.S. Application Data
(60) Provisional application No. 60/863,015, filed on Oct. 26, 2006.

(30) Foreign Application Priority Data
Mar. 29, 2006 (US) 11277845
Mar. 29, 2006 (US) PCT/US2006/011397
Oct. 26, 2006 (US) PCT/US2006/060271

(51) Int. Cl. A61K 9/127; A61K 38/06; A61K 33/38; A61P 3/00

(52) U.S. Cl. 424/450; 514/18; 424/618

(57) ABSTRACT

The invention enables management of mammalian disease related to decreased energy production in the mitochondria, the powerhouse of the cell. The invention uses the combination of liposomal reduced glutathione and l-arginine to increase the ability weight loss in individuals with excess weight. The mechanism of weight loss appears to be related to improving the inefficient production of energy by the respiratory transport chain of mitochondria, the function of which are influenced positively by the availability of antioxidant nitric oxide in a non-oxidized environment. This invention enables weight loss in individuals who's inability to lose weight is related to inefficiency of the biochemical pathways facilitating mitochondrial function and energy production. The pathways related to inability to lose weight are also related to the phenomenon of the inability to metabolize fats, which results in insulin resistance and diabetes. The invention is useful for the management of the metabolic syndrome. The metabolic syndrome is actually a group of metabolic factors associated with an increased risk of vascular disease problems. The invention is also useful for the resolution of fatigue that accompanies both weight gain and illnesses. The ability of the invention to increase the production of the biochemical agmatine in the central nervous system as well generally in the body is part of the benefit of the combination of liposomal reduced glutathione and l-arginine. In addition, the biochemical pathways stimulated by this invention can have a beneficial effect in individuals suffering from a variety of infectious diseases.

### CONTINUATION DATA

This application claims benefit of the filing of prior applications of this inventor and applicant: PCT US06/11397 and U.S. application Ser. No. 11/277,845 entitled “Administration of Glutathione (Reduced) via Intravenous or Encapsulated in Liposome for Treatment of TNF-alpha Effects and Flu-Like Viral Symptoms” both filed on Mar. 29, 2006, and U.S. Provisional Appl. 60/863,015 and PCT US06/60271 filed Oct. 26, 2006 and entitled “Liposomally Encapsulated Reduced Glutathione, Including With Other Pharmacologic Preparation, Capable Of Administration As An Oral, Topical, Intraoral Or Transmucosal Preparation For Reversal And Prevention Of Oxidation Of Cholesterol And Of Low Density Lipoprotein,” each of which is adopted by reference and incorporated into this application.

### FIELD OF INVENTION

This invention proposes the use of glutathione in the reduced state in a liposome alone or in combination with l-arginine, including in liposomal form, for treatment of inefficiencies in energy metabolism in the mitochondria leading to weight gain. The combination of liposomal glutathione and l-arginine can be used to manipulate the level of NO to stimulate mitochondrial biogenesis. This method is also useful in the management of some disease states related to mitochondrial dysfunctions as well infectious diseases.

### SUMMARY OF INVENTION

The invention enables management of, and the associated method of management of, mammalian disease related to decreased energy production in the mitochondria, the powerhouse of the cell. The invention uses the surprising finding that ingesting the combination of liposomal reduced glutathione and l-arginine results in weight loss in individuals using the combination for management of high blood pressure. The mechanism of weight loss appears to be related to inefficient production of energy by the respiratory transport chain of mitochondria, the function of which are influenced positively by the availability of antioxidant nitric oxide in a non-oxidized environment. This invention enables weight loss in individuals who's inability to lose weight is related to inefficiency of the biochemical pathways facilitating mitochondrial function and energy production. The pathways related to inability to lose weight are also related to the phenomenon of the inability to metabolize fats, which results in insulin resistance and diabetes. The invention is useful for the management of the metabolic syndrome. The metabolic syndrome is actually a group of metabolic factors associated with an increased risk of vascular disease problems. The invention is also useful for the resolution of fatigue that accompanies both weight gain and illnesses. In addition, the biochemical pathways stimulated by this invention can have a beneficial effect in individuals suffering from a variety of infectious diseases.

### SUMMARY OF DESCRIPTION

It is proposed that the continued usage of the present invention, liposomal glutathione with l-arginine will maintain function of the energy producing mitochondrial system of the body at a rate that will allow fat metabolism to occur and for weight to be lost. The continuous daily ingestion of the invention will provide the combination of adequate antioxidant protection and NO formation that is needed for the mitochondria to utilize fats efficiently and to allow the individual to lose weight. The increase in mitochondrial function enhanced by the mitochondrial biogenesis stimulated by this product will also improve lipid metabolism and diminish the likelihood of developing type 2 diabetes. The continuous daily ingestion of the current invention is proposed as a treatment for those individuals at risk of type 2 diabetes or those who are considered “pre-diabetic” by virtue of increased weight or family history or who are displaying the factors associated with “metabolic syndrome”.

### BACKGROUND

The expression of the enzyme endothelial Nitric Oxide synthase or eNOS that produces nitric oxide (NO) appear to be a critical factor in a number of cell functions ranging from arterial relaxation to an increase in the number and function of mitochondria, the sites of energy production in the cell. Factors that diminish the function of eNOS can have a significant impact on the function of cells and thus, the whole system. Human disease dysfunctions that are associated with diminished function of NO include hypertension and atherosclerosis. The role of NO in hypertension and atherosclerosis was reviewed in Guilford Patent Application # U.S. 60/863,015; PCT\US06\60271, and focused on the need for the use of reduced glutathione supplied in a liposome encapsulation in combination with L-arginine as a source for generating NO for the treatment of hypertension. As NO is a gas with a half-life in tissues estimated to be 5.6 seconds¹, it has not been possible to measure NO production in tissues directly and indirect measures have been used to monitor NO production. These indirect measures include monitoring the metabolic products of NO metabolism and of the physiologic effect of NO production. These physiologic effects include vascular smooth muscle relaxation resulting in lowering of blood pressure². In monitoring the individuals whose case examples were used in the Guilford Patent Application No. U.S. 60/863,015 and PCT\US06\60271, a completely unexpected and surprising additional effect has been observed. The effect is weight loss in the individuals using the combination of liposomal glutathione and arginine. Both of the individuals had excess weight and had been on methods of eating that were designed to help lose weight, but had not been able to accomplish weight loss. After starting the present invention, the two individuals noticed that they began to lose weight and found that it was easier to avoid eating excess amounts of food. An important, novel and surprising feature of the proposed invention is its use for facilitating weight loss and weight control.

An for the observed weight loss focuses on the relationship of mitochondrial function, glutathione and NO in and their interaction with the inflammatory immune hormones, called cytokines, that are known as the Tumor Necrosis Factor family and particularly Tumor Necrosis Factor-alpha (TNF-α). TNF-α regulates many biologic functions in the body ranging from organ development to immune homeostasis and disease³. Regulation of TNF-α is important because of the diverse impacts that it can have on different tissues. While on the one hand, TNF-α is essential for the host defense against infection, while on the other hand, TNF-α may have detrimental effects on tissues if not regulated properly³. For example, TNF-α is involved with the pathogenesis of multiple diseases including inflammation, obesity and insulin resistance^{4 5, 6}. A direct correlation between the genetic expression of TNF-α and insulin resistance has been observed^{7, 8}. TNF-α has been found to play a significant role in down-regulating the expression of eNOS, which then leads to a decrease in mitochondrial biogenesis and subsequent obesity⁹.

A review of the use of liposomal glutathione to ameliorate the effects of TNF-α exemplified by the management of viral disease is reviewed in Guilford U.S. patent application Ser. No. 11/420,168 filed Mar. 29, 2006 titled Administration of glutathione reduced via intravenous or encapsulated in liposome for the amelioration of the TNF-alpha effects and flu-like effects and flu-like viral symptoms and treatment and prevention of virus TNF-α factor is an inflammatory cytokine that causes damage by generation of oxidative stress.

TNF-α has been shown to sensitize cells and mitochondria to injury from peroxide (H₂O₂). Peroxide is an oxidant produced by various cells responding to viral infection including macrophage polymorphonuclear cells, natural killer (NK) cells and T-killer cells. Peroxide is a natural product of mitochondrial respiration but sensitization to H₂O₂ would be undesirable because of its biological destabilization. During aging there is an increased production of H₂O₂ in the liver mitochondria of many animal cells¹⁰. H₂O₂ is a product of superoxide radical dismutation that occurs in the mitochondria and is possibly related to damage of the mitochondria¹⁰.

It has been demonstrated that inflammatory related cells such as macrophages are accumulated in patches in the expanding adipose tissue^{11, 12, 13} with an increased release of inflammatory mediators, including TNF-α and iNOS^{10, 12}. It has been observed that upregulation of iNOS (which is induced in inflammatory conditions) often correlates with downregulation of eNOS¹⁴. Corroborating this, TNF-α increases iNOS expression in different cells and tissues including fat and muscle¹⁵. Recently it has been demonstrated that TNF-α can positively autoregulate its own biosynthesis in adipose tissue, contributing to the maintenance of elevated TNF-α in obesity¹⁶. In addition, stimulation of inflammatory responses has been observed in obese individuals with the finding of increased levels of the systemic inflammatory marker C-reactive protein (CRP)¹⁷.

A variety of stimuli can raise the level of TNF-α systemically or in specific tissues. These stimuli include bacterial or fungal exposure⁹, as well as hyperglycemia¹⁸. Environmental factors such as toxins⁹ including mercury cadmium, which are known to target mitochondria directly¹⁹ and lead, which lead will increase the amount of TNF-α that is released by subsequent exposure to lipopolysaccharide (LPS)²⁰. Of note is the fact that TNF-α generated at sites distant from organs can effect damage at organ sites such as liver²⁰.

The presence of TNF-α even in low concentrations increases the permeability of cells to damage from H₂O₂ peroxidation²¹. Under normal conditions the electron transport chain of mitochondria is the primary producer of the superoxide anion, which is precursor to other highly reactive species such as hydrogen peroxide and the hydroxyl radical^{22 23}. Glutathione (GSH) in mitochondria is the only defense available to metabolize hydrogen peroxide²⁴. The presence of TNF-α accelerates the membrane damage from peroxyl radicals and increases the demand and need for protection by glutathione. The amount of reduced glutathione contained in cells has been shown to be decreased in a concentration-dependent fashion upon exposure to TNF-α²¹. It appears that TNF-α decreases the availability of reduced glutathione, resulting in an increase in local oxidation stress. The formation of the oxidized form of glutathione, GSSG, can accumulate when its rate of formation exceeds the cells ability to convert it back to reduced glutathione, GSH. GSSG will be extruded from cells, resulting in an overall lack of reduced glutathione. It was also observed that GSH repletion inhibited the increased sensitivity of the TNF-α-treated endothelial cells to H₂O₂²¹. Thus, in the situation where there is increased oxidative stress and TNF-α in the mitochondria and the cell, an outside source of glutathione is useful in maintaining the antioxidant/oxidative stress balance (redox balance) in the cell and mitochondria.

TNF-α is overproduced in adipose and muscle tissues of obese individuals^{7 8 11 12} and plays a significant role in the development of obesity by diminishing eNOS expression and thus, decreasing NO production⁹. The presence of TNF-α markedly decreases both eNOS expression and mitochondrial biogenesis in cultured fat and muscle cells²¹. The present invention has the capacity to reverse this effect by supply reduced glutathione, the critical component for neutralizing the effect of TNF-α and at the same time supplies also l-arginine, which is needed to stimulate the availability of NO. For the function of the present invention, it is critical that the glutathione be available in the cell in a reduced form and this is done by using a liposomal formulation. Supplying arginine alone does not result in an efficient response as the presence of oxidative stress increases the likelihood that peroxynitrites will be formed from the production of nitric oxide in this situation. Thus, the combination in the present material provides an efficient mechanism for reversing the effect of TNF-α on fat and muscle cells. To be effective the liposomal reduced glutathione and arginine can be administered at the same time as liposomal glutathione plus oral arginine in capsules as outlined in Example 1, or as a combination of reduced glutathione and arginine in a liquid drink containing reduced glutathione and arginine in liposomes as in Example 2 or a gel cap containing liposomes with glutathione and arginine as in Example 3.

The use of the combination of liposomal reduced glutathione and L-arginine to induce loss of weight has not previously been reported. The use of the invention to moderate hypertension by increasing the production of NO the vasodilating biochemical S-nitrosylated glutathione (GSNO) is reviewed in Guilford Patent Application No. US 60/863,015 (as yet unpublished); PCT\US06\60271 (as yet unpublished). It has been demonstrated that GSNO is formed and found in mitochondria²⁵, but an impact on the functional effect of GSNO on mitochondria has not been demonstrated.

A significant factor in the loss of weight that accompanies the present invention is most likely due to reactivation of energy production through mitochondrial biogenesis. It is surprising to find this effect as the molecule GSNO has been shown to decrease the function of the oxidative phosphorylation pathway by binding to complex IV of the respiratory chain in the mitochondria. A study has demonstrates that GSNO reversibly inhibits oxygen utilization by attaching to cytochrome c at the end of the respiratory chain²⁶. In the Cleeter study mitochondria were isolated from rat gastroenemius muscle and their oxygen utilization measured in support media using a micro oxygen electrode and polarographic analysis of the metabolism of intact, whole mitochondria.²⁶. A reasonably skilled practitioner would assume therefore, that upregulation of GSNO would interfere with weight loss and inhibit mitochondrial respiration²⁵. Notwithstanding that apparent conclusion from the Cleeter study, which teaches upregulation of GSNO should be undesirable, independently, the inventor had commissioned another study. That unpublished study of “The effect of liposomal glutathione on the oxidation of the cholesterol components known as Low density lipoprotein (LDL) and high density lipoprotein (HDL)” was performed by Professor Michael Aviram The Lipid Research Laboratory Rambam Medical Center, Haifa Israel. I showed surprising results which led to conclusions by the inventor and gave rise to this invention. The study is described fully in PCT US06/60271 and U.S. Provisional 60/863,015. In reviewing the results of the unpublished Aviram study, it appears that LDL and HDL contain both the enzyme glutathione peroxidase (GPx) and it specific substrate reduced glutathione. The presence of GPx associated with LDL has not previously been reported. Thus the native lipids, as obtained from human subjects contain the mechanism to maintain defense against oxidants and to maintain a non-oxidized state. When materials known to cause oxidation are added to this system, there is a brief resistance to oxidation, but when the native glutathione is used up oxLDL is created. The surprising finding that leads to this invention is that the addition of even a small amount, μg/mL, of the liposomal encapsulated reduced glutathione results in a prolonged stabilization of the lipids against the oxidizers. The addition of 2 μg/mL Liposomal Glutathione to HDL resulted in prolongation of the lag time from 16 minutes in control HDL (incubated with no additions) up to 92 minutes observed for HDL that was incubated in the presence of Liposomal Glutathione.

The inventor concluded that the use of his prior invention upregulates GSNO, but contrary to the Cleeter study, in fact determined that his invention beneficially upregulated the GSNO.

Again, notwithstanding the conclusion of the Cleeter study, the inventor has discovered that this invention which had a combination to increase the up-regulation of GSNO, in fact is surprisingly beneficial for weight loss.

It is additionally surprising to find that the ingestion of the combination of liposomal reduced glutathione and l-arginine results in the benefit of weight loss as it is likely that the combination results in the production of NO in mitochondria. Studies have suggested that increasing NO will increase oxidative stress in mitochondria and inhibit key enzymes in a fashion similar to hydrogen peroxide (H₂O₂)²⁷. Inhibition of enzymes related to the Krebs cycle and ATP production have been thought to lead to an inhibitory effect on the respiratory chain 28, which would lead to the expectation that increasing NO availability to mitochondria would result in a decrease of metabolic function and an increase in weight²⁷, not an increase in metabolic function and decreased weight as reported in Case Examples 1 and 2. Mitochondrial dysfunction has been shown to be related to the pathophysiology of obesity in gene array studies of both animals and humans as many genes encoding for mitochondrial proteins are inversely correlated with body mass^{29 9}.

Obesity can be defined as the condition in which the natural energy reserve stored in the fatty tissue of humans is increased to the point that it is associated with health abnormalities or even mortality. In simple terms, obesity is the accumulation of excess amounts of fat which can become so enlarged that it restricts the ability of the individual to move around. Internally, obesity is associated with accumulation of fat in tissues such as the liver, to the point that the liver function is compromised. This condition is known as non-alcoholic fatty liver. A method of determining an indication of an individual's level fatness can be calculated from the relationship of their weight to their height and is known as the body mass index (BMI). The BMI is calculated by the formula BMI=Weight (pounds)/Height (inches)².

BMI Categories:
Underweight=<18.5
Normal weight=18.5-24.9
Overweight=25-29.9
Obesity=BMI of 30 or greater

Obesity is also associated a group of risk factors of heart disease that have become known as the metabolic syndrome. These risks factors include
The excessive fat tissue in and around the abdomen which is also known as abdominal obesity
Abnormalities of the lipids in the blood including low HDL cholesterol, high LDL cholesterol and high triglycerides that are associated with the formation of atherosclerotic plaque in artery walls
Elevated blood pressure
Insulin resistance or the inability to utilize glucose properly
Pro-inflammatory states, that is the presence of proteins in the blood indicating inflammation in the body and typified by the elevation of C-reactive protein in the blood.
Increased tendency to form clots in the blood called the prothrombotic state accompanied by high fibrinogen or plasminogen activator-1 in the blood.
   People with the metabolic syndrome carry an increased risk of coronary heart disease and other vascular diseases such as stroke and peripheral vascular disease related to decreased flow of blood to vital tissues as well as to type 2 diabetes.

The combination of obesity and diabetes is increasing at a rate that is called epidemic. The CDC reports that since 1980 the incidence of obesity has gone from 15% of the population to 33% in 2004. The incidence of obesity in children is also increasing with the prevalence now estimated at 17.4% (http://www.cdc.gov/nccdphp/dnpa/obesity/, viewed Mar. 5, 2007) to 25%³⁰. World wide it is estimated that 1.1 billion adults and 10% of the children are classified as overweight or obese³¹.

Type 2 diabetes is the most common chronic metabolic disease in the elderly, affecting ˜30 million individuals 65 years of age or older in developed countries³². The estimated economic burden of diabetes in the United States is ˜$100 billion per year, of which a substantial proportion can be attributed to persons with type 2 diabetes in the elderly age group³³. At the same time, obesity has reached epidemic proportions in developed countries. While most experts view the cause of obesity to be related to overeating and a sedentary life style, the biochemistry of obesity is pointing to changes in the fundamentals of energy metabolism at the most basic levels, the Krebs cycle and the oxidation of fats²⁷, both of which occur in the mitochondria of the cell. It appears that the lack of energy and decreased ATP production drives the appetite in a search for energy sources²⁷. In this model, fatigue is viewed as a decrease in available energy sources such as ATP (Green) and is associated with increased appetite and decreased exercise capacity⁹ due to decreased energy. There is a line of research that suggests that aging is associated with a reduced capacity for oxidative phosphorylation in muscle which is thought to be due to a decline in mitochondrial function or number³⁴. At the biochemical level, aging, type 2 diabetes and obesity seem to have common factors. At the genetic level there are a number of factors related to mitochondrial function that come into play include genes related to nuclear respiratory factor (NRF)-dependent transcription and the expression of peroxisomal proliferator activator receptor γ coactivator (PGCI)α and -β (PPARGCI and PERC), coactivators of both PPARG and NRF-dependent transcription. These respiratory chain factors have been found deficient in both pre-diabetic and diabetic subjects Patti³⁵. The decrease in PGC-1 seems to be a common factor in insulin resistance and diabetes mellitus.

A metabolic cycle becomes established in obesity that causes a persisting gain of weight. Briefly, interruption of the Krebs cycle at the level of the utilization of the aconitase enzyme results in a decrease in the production of ATP in TCA cycle by preventing citrate to isocitrate conversion. The increase in citrate activates acetyl-CoA carboxylase (ACC), the committing step in fatty acid synthesis. Thus, inhibition of aconitase diverts metabolism from energy production to energy storage. The readier is referred to the article by Wlodek²⁷ for a detailed review of this metabolism. The summary is that an inflammatory response leads to TNF-α release, stimulation of IL-1 and oxidation stress. These factors inhibit the Tri-carboxylic acid cycle (Krebs cycle), lowering energy production and increasing fat synthesis. At the same time, adipose tissue has been shown to be the target of inflammatory cells and there is a increased release of TNF-α and IL-1^{36 8}. This process sets up a repeating cycle leading to obesity. The present invention is a combination that breaks the pattern of the metabolic cycle leading to obesity. A reasonably skilled practitioner would assume therefore, that increased production of NO would interfere with weight loss and inhibit mitochondrial respiration as discussed by Wlodek²⁷. Notwithstanding that apparent conclusion, which teaches increased production of NO in mitochondria should be undesirable, the inventor has discovered that this invention which had a combination to increase production of NO in fact is surprisingly beneficial for weight loss.

Diminished mitochondrial function in muscle appears to be a common thread linking aging, obesity and type 2 diabetes. It appears that while there is an overall reduction in electron transport chain activity in type 2 diabetes and obesity, there is a striking deficiency of sub-sarcolemmal mitochondria observed in type 2 diabetes³⁷. It has been observed that insulin infusion increases muscle ATP production and mitochondrial protein synthesis in Ican volunteers, but not in type 2 diabetic subjects³⁸. These findings have recently been confirmed in elderly subjects by non-invasive technology using magnetic resonance spectroscopy³⁹. As mitochondrial biogenesis can be stimulated regardless of age⁴⁰ as demonstrated by methods such as aerobic exercise³⁴, the use of the present invention represents a combination and method of increasing mitochondrial biogenesis, reducing adipose formation and decreasing the likelihood of developing insulin resistance at any age.

### Reactive Species of Oxygen and Nitrogen

In the discussion of mitochondrial function and the utilization of glutathione, a discussion of free radical terminology is needed.

The hydroxyl radical is one of a group of radicals that are formed from reactions with oxygen. The oxygen molecule is a stable diradical that can be represented •O—O• and is a stable molecule with the hyphen representing a single bond and the “dots” representing electrons available for pairing and bonding. The most familiar radical reaction with oxygen is combustion, or burning. Combustion is comprised of a chain reaction of radicals. In the case of oxygen, after enough energy has been entered into the system, the stable radical •O—O• is converted to singlet oxygen radicals (O•⁻). Inside the body, the reactions with oxygen are limited by the relatively low amount of oxygen, the presence of materials that prevent the reaction from proceeding and the relatively low temperature of the body. However, singlet oxygen can be formed from O₂ by enzymes that initiate this reaction. In the body, interactions with enzymes and other donors of electrons such as transition metals leads to the formation of a number of variations of from diradical oxygen (•O—O•).

The presence of a transition metal such as iron in the ferrous state combined with peroxide, found in many biochemical reactions in the cell in results in a reaction called the Fenton Reaction

Fe²¹+H₂O₂→Fe³¹+OH•+OH³¹

Resulting in the formation of 2 hydroxyl radicals, or strictly speaking, as illustrated a hydroxyl radical and a hydroxide ion.
Other methods of forming peroxide in the cell include
O2-+e [symbol] O2•⁻ Superoxide anion
O2 HO [symbol] HO•⁻+OH— Hydroperoxyl radical
HO+e-+H+-->H₂O₂ Hydrogen peroxide
H2O2+e---->•OH+OH— Hydroxyl radical
Because it is involved in the generation of free radicals H₂O₂ is included in the general term reactive oxygen species.
Another well known free radical that is produced during normal physiological processes, this is nitric oxide (NO). Nitric oxide is produced by the vascular endothelium and is responsible for relaxation of both arteries and bronchial tubes, the airways in the lung.
The interaction of the superoxide molecule, with nitric oxide as follows:

O₂•⁻+NO [symbol] ONOO•⁻-(PEROXYNITRITE),

Peroxynitrate is a strong oxidant and contributes to damage of cells, especially in the lining of arteries and airways. Excess (ONOO•⁻) may be produced when cytokines have increased production of both (NO) and (O2•⁻). At physiological pH peroxynitrate causes direct damage to proteins, and decomposes into toxic products that include nitrogen dioxide and hydroxyl radicals.
The list of potential radicals from oxygen and nitric oxide include:

| Primary Reaction | Oxygen species Configured |
|---|---|
| Oxygen | (•O—O•) |
| Superoxide Anion | (O₂•⁻) |
| Hydroxyl Radicals | (OH•⁻) |
| Hydrogen Peroxide | (H₂O₂) |
| Singlet Oxygen | (O•⁻) |
| Nitric Oxide | (NO—) |
| Peroxynitrite | (ONOO•⁻) |

The interaction of these free radicals with alkyl groups such as those on proteins and lipids produce secondary reactions

| | |
|---|---|
| Peroxyl Radical | (ROO•⁻) |
| Alkoxyl Radical | (RO•⁻) |

Alky radicals can also bond together to form compounds called polymers. Lipoproteins can be considered radicals as they are considered polymers of amino acids with a fatty acid end group.

These highly reactive radical species of oxygen are also referred to as reactive oxygen species and abbreviated ROS. The highly reactive radical species of nitric oxide are called reactive nitrogen species (RNS). Both Reactive oxygen species (ROS) and reactive nitrogen species (RNS) are important mediators of cell and tissue injury (see figs.), and are major players in the process of aging and apoptosis, a mechanism of cell death.

Thus oxygen-derived free radicals—superoxide anion, (O2•⁻), hydroxyl radicals OH•⁻ or metabolites such as hydrogen peroxide and hypochlorous acid (HOCl) must be regulated.

When superoxide anions are formed, they are removed rapidly by interaction with an enzyme called superoxide dismutase. Removal of hydroxyl radicals require interaction with an antioxidant called glutathione (Wu).

For the removal of the OH•⁻ radical, the antioxidant molecule glutathione, which is abbreviated GSH, loses the hydrogen atom to OH•⁻, creating HOH and the radical GS•⁻.

2OH•⁻+2GSH→2H₂O+GSSG

The GSH is the reduced form of glutathione and GSSG is the oxidized form⁴.

### Ionizing and Non-Ionizing Radiation

Radiation that has enough energy to move atoms in a molecule around or cause them to vibrate, but not enough to remove electrons, is referred to as “non-ionizing radiation.” Examples of this kind of radiation are sound waves, visible light, and microwaves.

Radiation that falls within the “ionizing radiation” range has enough energy to remove tightly bound electrons from atoms, thus creating ions. This is the type of radiation that people usually think of as ‘radiation.’ We take advantage of its properties to generate electric power, to kill cancer cells, and in many manufacturing processes. Higher frequency ultraviolet radiation begins to have enough energy to break chemical bonds. X-ray and gamma ray radiation, which are at the upper end of magnetic radiation, have very high frequency—in the range of 100 billion billion Hertz—and very short wavelengths—1 million millionth of a meter. Radiation in this range has extremely high energy. It has enough energy to strip off electrons or, in the case of very high-energy radiation, break up the nucleus of atoms.

Ionization is the process in which a charged portion of a molecule (usually an electron) is given enough energy to break away from the atom. This process results in the formation of two charged particles or ions: the molecule with a net positive charge, and the free electron with a negative charge.

Each ionization releases approximately 33 electron volts (eV) of energy. Material surrounding the atom absorbs the energy. Compared to other types of radiation that may be absorbed, ionizing radiation deposits a large amount of energy into a small area. In fact, the 33 eV from one ionization is more than enough energy to disrupt the chemical bond between two carbon atoms. All ionizing radiation is capable, directly or indirectly, of removing electrons from most molecules. There are three main kinds of ionizing radiation: alpha particles, which include two protons and two neutrons; beta particles, which are essentially electrons; and gamma rays and x-rays, which are pure energy (photons).

The majority of radiation injury in cells depends on oxidative stress. Irradiation and absorbed doses, duration of the irradiation and the susceptibility of the tissue against radiation are the factors that cause variations on living cells⁴². Mitochondrial GSH becomes critically important against ROS-mediated damage because it not only functions as a potent antioxidant but is also required for the activities of mitochondrial glutathione peroxidase and mitochondrial phospholipid hydroperoxide glutathione peroxidase⁴³ which removes mitochondrial peroxides.

Because the mechanism of this invention has implications in mitochondrial function, and relates to oxidative stress, the invention has implications for what otherwise might seem to be unrelated fields, those fields being weight loss and radiation or chemotherapy. The common link between these fields is the symptom of fatigue that is related to decreased mitochondrial function.

### Protection of Mitochondria Against Oxidative Stress

It appears that NO competes competitively with oxygen for binding on the cytochrome oxidase enzyme, complex IV²⁸, which would regulate the utilization of oxygen and the function of the electron transport chain in forming ATP (Haynes). More recently, it has been shown that the binding to cytochrome c oxidase is at the copper group of cytochrome c oxidase, complex IV of the mitochondrial respiratory chain⁴⁴. This information suggests that the presence of NO appears to have an inhibitory effect on mitochondrial oxidative phosphorylation⁴⁵⁻⁴⁸ NO can also have an effect on the first complex (complex I) in the respiratory chain. It appears that while the inhibition of complex IV is reversible, prolonged exposure of complex I to nitric oxide will result in a persisting inhibition⁴⁷. However, it appears that the NO inhibition of complex I can be reversed by the introduction of reduced glutathione⁴⁷. Clementi⁴⁷ notes that as reduced glutathione diminishes within mitochondria, the inhibition of complex I increases proportionally⁴⁷. The biochemistry of how glutathione protects complex I is not clear, although it may be due to scavenging of nitrositive species or by direct removal of NO with the formation of GSNO⁴⁷. It appears that the interaction of NO and GSH to form GSNO may be a built in protective mechanism that protects the mammalian cell against nitrositive stress that could cause host cell damage when increased generation and release of NO occurs. NO production is increased during potentially oxidative stress events such as during defense against invading microorganisms. GSH would also protect the local complex against peroxynitrite that may be formed at the site of complex I⁴⁷ as it has been reported that GSH converts the peroxynitrite radical (ONOO—) into S-nitrosyl glutathione (GSNO)^{49 50}. It appears that the amount of oxygen available to the cell regulates the formation of NO, as hypoxia increases both Ca²⁺ influx and NO synthesis, suggesting that as the concentration of oxygen in the environment decreases, the cell adapts itself by reducing its respiratory rate, and thus its oxygen requirement⁴⁸.

NO regulation of OXPHOS by competing with oxygen for cytochrome c oxidase function leads to regulation of activity with low oxygen tissue environments and to regulation as part of adaptive responses to stress such as that seen with alcohol toxicity and hypoxia. The presence of mtNOS allows the mitochondria to self regulate OXPHOS^{51 52 53}.

The observation by Clecter²⁶ suggests that the molecule GSNO reversibly inhibits oxygen utilization by attaching to cytochrome c at the end of the respiratory chain. This suggests that GSNO is inhibitory to mitochondrial function and teaches away from the observations made in this application.

An additional problem develops in non-functioning mitochondria. Cytochrome c oxidase has been shown to have some reductive capacity in removing peroxynitrite, ONOO⁻. Nitric oxide has been found to have a Janus-faced role in regard to endothelial function in that NO is needed for vasodilation and the prevention of hypertension, but in the presence of oxidative stress NO becomes a source of the cell damaging peroxynitrite radical. Normally functioning mitochondria have several methods for preventing peroxynitrite accumulation, however, if cytochrome c oxidase is not functioning normally, the respiratory chain no longer has the interaction with oxygen available and large amount of superoxide, O₂⁻, can be formed⁵⁴. The loss of cytochrome c oxidase function also leaves more O₂ available to stimulate mtNOS to form more NO. The sustained production of peroxynitrite, stimulates demand for glutathione⁵⁵ and there is evidence that peroxynitrite can be scavenged by glutathione⁵⁶, ⁵⁷, ⁵⁸. It has been reported that GSH converts the peroxynitrite radical (ONOO—) into GSNO⁵⁰, ⁴⁹. Thus, peroxynitrite formation requires a constant supply of glutathione or it will result in damage to cells as evidenced by the accumulation of peroxynitrite in damaged tissue⁵⁹, ⁶⁰. As reduced glutathione must be supplied from the surrounding cytosol, there is a constant demand for reduced glutathione. Although part of the oxidized form of glutathione, GSSG, that is formed can be reduced back to GSH through the action of glutathione reductase (GRD), it appears that this source of GSH is minor compared with GSH production dc novo and that the presence of other oxidative stresses such as oxLDL may limit the incorporation of substrates into the formation of GSH⁶¹.

The liposomal glutathione component of the current invention has been demonstrated to slow the progression of atherosclerosis in ApoE knockout mice, which are well characterized as the animal model for atherosclerosis, which has been reviewed in Guilford Patent Application # U.S. 60/863,015; PCT\US06\60271. The application also notes that while lowering oxidized LDL is a beneficial goal of liposomal glutathione additional benefit would accrue from the elevation of HDL and combining the liposomal glutathione with statin drugs was proposed. To facilitate the elevation of HDL it is now proposed that the liposomal glutathione be combined with one of a class of drugs known as Cholesteryl ester transfer protein (CETP). This protein transfer lipids in the form of cholesterol esters from HDL, which contains apoprotein-A (apo-A) to lipoproteins that contain apo-B such as very low density lipoprotein (VLDL) and LDL. Normally CETP also takes up one TriglycerideG molecule from LDL or VLDL and transfers it to HDL. A CETP inhibitor would thus be expected to raise plasma HDL cholesterol (HDLc) levels, lower LDL cholesterol (LDLc), and provide a potential therapeutic benefit for patients with coronary artery disease (CAD)⁶².

Recently one of the first of this potential class of lipid moderating agents, the cholesteryl-ester transfer protein inhibitor called torcetrapib passed phase trials for the management of low HDL. It has been subsequently found that torcetrapib has an increased association with an increase in death and heart problems compared to the control statin group. Additionally there is some elevation of blood pressure with torcetrapib. It is proposed that torcetrapib be combined with the present invention, liposomal glutathione and l-arginine to increase the beneficial response to the drug and to decrease the likelihood of side effects.

It has been thought that calorie restriction has been the only way to preserve mitochondria and extend life span in rodents⁶³. Recent article has confirmed that a 25% caloric deficit either by caloric restriction alone or by a combination of caloric restriction and exercise increased mitochondrial function in overweight, but non-obese humans⁶⁴ Calorie restriction has also been shown to delay the onset of a number of age related diseases including cancer, atherosclerosis and diabetes in rodents and possibly primates and even in humans⁶⁵. Recent evidence that calorie restriction increases the formation of eNOS mediated mitochondrial biogenesis⁶⁶ has focused the attention back onto the availability of NO in the mitochondria⁶⁷. Increasing the availability of NO results in a surge of NO that activates synthesis of a broad array of mitochondrial proteins and increases product of mtDNA, respiratory chain function, and ATP levels in a variety of tissues including brain, liver and heart⁶⁶. Of additional note is that increasing evidence suggests that SIRT1, the mammalian ortholog of the SIR2 gene, a member of the SIR (silent information regulator) genes that mediates the life-extending effect of calorie restriction in yeast is also up-regulated⁶⁶ and may contribute to longevity of organisms through a variety of effects⁶⁷.

The purpose of this invention is to facilitate the proper mediation of upregulation of NO.

These observations are leading to the concept that increased availability of NO, in this model by using calorie restriction to increase eNOS, results in mitochondrial biogenesis by increased PGC-1αexpression and upregulation of SIRT1 and similar longevity promoting agents⁶⁷. There are suggestions that SIRT1 may mediate mitochondrial biogenesis in fat cells by increasing PGC-1α, which coordinates the genes involved not only with mitochondrial biogenesis, but also oxidation of fatty acids⁶⁸ and decreases adipose tissue formation

It appears that increased presence of NO stimulates mitochondrial biogenesis with an accompanying set of proteins that not only stimulate mitochondrial reproduction, but also protect and repair mitochondrial DNA. Thus, NO has the ability to reduce fat accumulation by oxidation of fatty acids, lipolysis and inhibition of adipocyte formation by stimulating SIRT1, PGC-1α and mitochondrial biogenesis⁶⁷ Nisoli points out that up to this point in time, this effect has been accomplished only by calorie restriction⁶⁷.

This application proposes the use of the invention, liposomal encapsulation of reduced glutathione with liposomal encapsulated l-arginine or the contemporaneous ingestion of l-arginine to increase the production of NO for the stimulation of biogenesis of mitochondria and the improved oxidation of fatty acids to result in weight loss. The mechanism appears to be through the pathway described by Valerio in which NO production induces mitochondrial biogenesis, with a concomitant increase of PGC-1α, NRF-1, and Tfam gene expression, oxygen consumption, and ATP production in adipose and muscle cells⁹. Conversely, in the absence of NO production, a lack of mitochondrial biogenesis results in visceral and skeletal obesity, increased muscle fat accumulation and metabolic syndrome⁹. Indeed, a human study has shown that there is an inverse correlation between skeletal muscle eNOS content (the source of NO), the percent body fat and the body mass index in young adult women⁶⁹

Muscle activity is dependent on a steady flow of ATP. ATP allows muscle to get into the position where the elongated myosin is able to contract, shortening the muscle. The “ready to contract” state appears as muscle relaxation. The energy is stored in the biochemical component of actin and myosin. An analogy suggests that ATP provides the energy to pull back the trigger, with this situation storing the energy until the muscle contracts. Thus, it can be said that energy from ATP is required for muscle relaxation⁷⁰. Skeletal muscle has a high reliance on OXPHOS³⁷ and skeletal muscle becomes the focus of biochemical defects related to glucose metabolism in obesity as abnormal metabolism of fatty acids is found in obesity-related insulin resistance⁷¹

The net result of decreased energy production for the individual is the perception that even though they may have recently eaten, they have the perception of needing more energy and thus fell hungry for more food⁹. At the same time that they are increasing food to provide energy, obese patients feel less energetic and decrease their physical activity in order to conserve energy²⁷ Caloric restriction has been shown in mice to increase eNOS and mitochondrial biogenesis; however in obese humans it has been observed that a restrictive diet lowers the already deficient oxidation of lipids It is likely that even if the individual were able to lose weight, if the mitochondrial function were not corrected, that they would be likely to regain the weight very quickly after stopping a restrictive diet. The deficit in mitochondrial function would explain the continuing cycles of weight gain following weight loss that is experience by many individuals on restrictive diets. It is probable that the lack of NO stimulated mitochondrial biogenesis is the underlying cause of the inability to metabolize appropriately in the obese individual.⁹

The present invention provides a surprising and unexpected combination for modulating the biochemical abnormalities associated with obesity. The liposomal glutathione provides neutralization for the effect of TNF alpha, and at the same time provides that ability to maintain the NO produced from arginine to be used efficiently either by providing the appropriate antioxidant environment to prevent the oxidation of NO or by binding NO into GSNO. GSNO potentially provides benefit in several ways such as providing a stable carrier of NO or by a direct action that has not been identified in mitochondria, but is well documented in vascular relaxation. The likelihood that GSNO provides a direct action on mitochondria is increased by the observation that it takes ATP to provide muscle relaxation, or stated another way, relaxation (of muscle) takes energy. For GSNO to provide relaxation in arterial vessel smooth muscle respiratory chain activity in the mitochondria must be present to provide the energy. Part of the surprise of the success of this combination is the observations that GSNO causes a defect in the respiratory chain function according to one study²⁶.

The present invention, liposomal glutathione provides the ability to restore mitochondrial biogenesis and a return to energy production that can result in weight loss as illustrated in case examples 1 and 2. In attention to the ability to lose weight the case examples also noted that they had an easier time avoiding “snacking” and the hunger for between meal snacks that they had experienced on previous attempts to lose weight. Thus, the present invention provides “appetite suppression” by providing the normal mechanism of appetite suppression, namely, the feedback that enough energy is being provided to the system. The present invention is proposed as an appetite suppressant.

An additional mechanism for appetite suppression is also presented by the present invention. The metabolism of arginine can follow several pathways. While the production of NO by the interaction of arginine and nitric oxide synthase is well known, a less well known metabolic pathway will convert arginine to the amino acid like biochemical agmatine (1-amino-4-guanidino-butane). Agmatine, which falls into the family of molecules known as polyamines such as putrescine, spermine, spermidine, which are formed from ornithine and are essential for the growth, the maintenance and the function of normal cells⁷². Agmatine however, is formed specifically from arginine⁷² by the decarboxylation of l-arginine by an enzyme known as arginine decarboxylase (ADC) Agmatine has subsequently been found to be widely distributed in mammalian tissues and both a hormone like action⁷³ as well as an action as a neurotransmitter⁷². Agmatine and the ADC enzyme have been found in rat brain, kidney, astrocytes, endothelium and vascular smooth muscle cells⁷⁴. In the brain agmatine is synthesized by the decarboxylase enzyme located in the mitochondria⁷⁵ of astrocytes and neurons⁷⁶ and interacts with receptors such as nicotine, N-methyl-D-aspartate (NMDA) receptor, benzodiazepine and intracellular imidazoline receptors. The molecule is transported into the matrix of mitochondria by an energy-dependent mechanism that seems to be specific for this molecule⁷⁷. Several functions have been associated with agmatine including stimulation of fatty acid oxidation in mitochondria⁷⁶ and blocking the N-methyl-D-aspartate (NMDA) receptor^{78 79}, the site of stimulation by glutamate, an excitatory toxin. Agmatine has been shown to potentiate morphine analgesia, reduced dependence/withdrawal from morphine⁸⁰ and attenuates symptoms of withdrawal from ethanol in a rat model⁸¹. The exact mechanism of the pain relieving action of agmatine has not been demonstrated, but the interactions with N-methyl-D-aspartate (NMDA) receptors, alpha2-adrenergic receptors, and intracellular cyclic adenosine monophosphate (cAMP) signaling have been proposed as possible explanations⁸⁰. The lack of penetration of agmatine into the brain has previously prevented the use of agmatine as a direct therapeutic agent⁸⁰.

It is proposed that the ingestion present invention liposomal glutathione combined with l-arginine is a combination that raises the level of agmatine both peripherally and centrally. As there is some question of the ability of agmatine to be absorbed across the blood brain barrier a combination that raises agmatine in the central nervous system offers a real advantage. The increase of agmatine interacts with imidazole receptors and mediates a sympatho-inhibitory action to lower blood pressure via a central nervous system action. In addition, agmatine has a peripheral activity related to increasing insulin secretion from beta cells and the ability of increasing lipid metabolism on fat cells. It is proposed that the present invention raises agmatine levels increasing the weight loss components of the invention. In addition, it is proposed that the stabilizing effect that agmatine has on withdrawal symptoms from both morphine and alcohol contribute to the ability to withdraw from excess amounts of food and contributes significantly to the appetite suppression quality of this invention. The combination of these actions is reviewed in Case Example 2.

Several references have been found for the use of the biochemical agmatine for the either alone or in combination with other materials for the treatment of various illnesses. However, no reference is found for the combination of liposomal glutathione and l-arginine to enhance the endogenous production and physiologic utilization of agmatine in the body.

Bajusz, et al. in U.S. Pat. No. 4,346,078 reference the use of agmatine derivatives for use as anticoagulant therapeutics. This patent does not reference the combination of liposomal glutathione and l-arginine to enhance the endogenous production and physiologic utilization of agmatine in the body.
Raisfeld in U.S. Pat. No. 4,507,321 references compositions containing agmatine for the use topically on epithelial cells to stimulate regrowth in situations such as wound healing, does not reference the combination of liposomal glutathione and l-arginine to enhance the endogenous production and physiologic utilization of agmatine in the body.
Lubec in U.S. Pat. No. 5,077,313 issued Dec. 31, 1991, references the use of arginine, spermidine, creatine, or agmatine in the treatment of glucose-mediated collagen cross-links in diabetes-mellitus patients. This patent does not reference the combination of liposomal glutathione and l-arginine to enhance the endogenous production and physiologic utilization of agmatine in the body.
Sjoerdsma et al. in U.S. Pat. No. 5,196,450 references the use of derivatives of arginine and agmatine, specifically this invention relates to certain agmatine and arginine derivatives which are enzyme inhibitors, which interrupt the biosynthesis of polyamines and which inhibit the growth of certain protozoans. These derivatives are intended for the treatment of parasitic infections in mammals. This patent does not reference the combination of liposomal glutathione and l-arginine to enhance the endogenous production and physiologic utilization of agmatine in the body.
Regunathan, et al. in U.S. Pat. No. 5,574,059 references the use of agmatine as an 1.sub.2 imidazoline receptor agonist to treat disorders mediated by vascular smooth muscle proliferation by administering a vascular smooth muscle antiproliferative substance. The disorders include atherosclerosis, risk of blockage of artery after coronary angioplasty or blood vessel injury from non-angioplasty cause, and proliferative diabetic retinopathy. 1.sub.2 imidazoline receptor agonists include idazoxan, UK 14,304, naphazoline, cirazoline and agmatine. This patent refers to the administration of agmatine and does not reference the combination of liposomal glutathione and l-arginine to enhance the endogenous production and physiologic utilization of agmatine in the body.
Gilad, et al. in U.S. Pat. Nos. 5,677,349 and 6,114,392 references the use of agmatine or derivatives of agmatine, in the treatment of acute neurotrauma (such as stroke) and degenerative disorders of the central and peripheral nervous system (such as dementia). This patent does not reference the combination of liposomal glutathione and l-arginine to enhance the endogenous production and physiologic utilization of agmatine in the body.
Szelke, et al in U.S. Pat. No. 6,096,712 reference Kininogenase inhibiting peptides or peptide analogues with C-terminal related to agmatine or noragmatine. The compounds are intended for the treatment of a variety of disease states related to inflammation and hypotension. This patent does not reference the combination of liposomal glutathione and l-arginine to enhance the endogenous production and physiologic utilization of agmatine in the body.
Fairbanks, et al in U.S. Pat. No. 6,150,419 reference the use of agmatine as treatment and composition for neuropathic pain. This is a continuing application of International Application PCT//US98/17033, with an international filing date of Aug. 17, 1998, which claims the benefit of U.S. Provisional Application No. 60/055,847, filed Aug. 15, 1997. This patent does not reference the combination of liposomal glutathione and l-arginine to enhance the endogenous production and physiologic utilization of agmatine in the body.
Bouyssou, et al. in U.S. Pat. No. 6,429,229 reference the use of objects salts of derivatives of amino acids using agmatine or arginine as examples in which keto acids and of amine derivatives, as well as their use for the preparation of pharmaceutical compositions for the treatment of pathologies in which are involved silent neurons. This patent does not reference the combination of liposomal glutathione and l-arginine to enhance the endogenous production and physiologic utilization of agmatine in the body.

Applicant the University of Kentucky Research Foundation applied for a PCT application published as WO2001/095897 entitled Agmatine and Agmatine Analogs in the Treatment of Epilepsy, Seizure, and Electroconvulsive Disorders, published Dec. 20, 2001. The application referred to pharmaceutical preparations containing of agmatine, congeners, analogs or derivatives thereof for use in preventing or treating epilepsy, seizures and other electroconvulsive disorders are provided. The application referenced embodiments including administering an effective amount of agmatine, an agmatine analog or a pharmaceutically acceptable salt thereof to a human subject in need of treatment or prevention of epilepsy, seizure or other electroconvulsive disorder to treat, reduce, or prevent the disorder in the subject.

While the application references the use of agmatine to treat epilepsy, but does not reference the combination of liposomal glutathione and l-arginine to enhance the endogenous production and physiologic utilization of agmatine in the body.

Applicant The Regents of the University of California filed a PCT Application published as WO 1998/013037 entitled Methods of Using Agmatine to Reduce Intracellular Polyamine levels and to Inhibit Inducible Nitric Oxide Synthase. That invention proposed a method of reducing polyamine levels intracellularly by administering an arginine derivative to a mammal and a pharmacological composition comprising agmatine in a physiologically acceptable buffer. The invention was described as “a method of treating conditions resulting from abnormally elevated intracellular polyamine levels by administering an arginine derivative or agmatine to the cells in condition such as cancer or hypertrophy. The present invention further provides a method of regulating inducible nitric oxide synthase while maintaining constitutive nitric oxide synthase, by administering agmatine or an arginine derivative to a mammal.”

However, WO 1998/013037 does not reference the combination of liposomal glutathione and l-arginine to enhance the endogenous production and physiologic utilization of agmatine in the body.

Applicant The Proctor & Gamble Company filed an application entitled the Regulation of Mammalian Hair Growth as WO2005/078157. However, that invention focused on a topical skin care composition “containing a safe and effective amount of a skin care active comprising agmatine, and its salt; a safe and effective amount of a first additional skin care active selected from the group consisting of BHT or BHA, hexamidine, cetyl pyridinium chloride, green tea catechins, phytosterols, ursolic acid, compounds derived from plant extracts, their salts and derivatives; and a dermatologically acceptable carrier for the agmatine composition.” The present invention also relates to methods of using such agmatine compositions to regulate hair growth and the condition of mammalian skin.

The application WO2005/078157 referred to a topical composition containing agmatine, but did not reference l-glutathione, much less liposomal glutathione in combination with agmatine or arginine. In addition the discussion of hair growth regulation does not relate to the subject of this invention. That application does not reference the combination of liposomal glutathione and arginine as a topical preparation for the stimulating the metabolism of fat under the skin surface.

Yet another PCT application, Wohlrab, J., “Use of Agmatine for Topical Application,’ WO 2003/092668 was published and referenced the use of agmatine and/or derivatives thereof and salts for topical application in therapy and prophylaxis of pathological alterations of the skin and/or for cosmetic use. The Wohlrab art did not reference the combination of liposomal glutathione and l-arginine.

Stohs et al, in US patent application 20060292134 reference the use of a composition of creatine, L-arginine-.alpha.-ketoglutarate, D-ribose, L-carnitine, L-citrulline, and pyruvate for enhancing cellular energy with increased ATP production and to increase muscle mass of the subject. There is no reference to the use of liposomal reduced glutathione in combination with arginine to increase cellular metabolism, to increase mitochondrial biogenesis or for weight loss.

Koide et al, in Patent Application 20060280776 reference the use of an omega-3 polyunsaturated fatty acid (PUFA) or an omega-6 PUFA and at least one of the following L-arginine, L-ornithine, an L-arginine precursor and an L-ornithine precursor, and further includes diacylglycerol, a middle or short chain fatty acid, a phytosterol, a nucleo-base, a nucleoside, a nucleic acid, dextrin, various vitamins, various minerals or a probiotics material. There is reference to the use of arginine to activate lipase, however, there is no reference to the use of liposomal reduced glutathione in combination with arginine to increase cellular metabolism, to increase mitochondrial biogenesis or for weight loss.

Ron, in Patent Application 20050288373 references the administration of arginine or time-release arginine for use in treating a variety of conditions including lowering triglyceride levels, inducing thermogenesis, weight loss and treatment and prevention of obesity and obesity related conditions, such as diabetes. There is no reference to the use of liposomal reduced glutathione in combination with arginine to increase cellular metabolism, to increase mitochondrial biogenesis or for weight loss.

Byrd in Patent Application 20050085498 references a formulation comprised of four active components which are a lipid soluble thiamine, lipoic acid, arginine, alpha.-ketoglutarate, and a creatine derivative for oral administration. There is no reference to the use of liposomal reduced glutathione in combination with arginine to increase cellular metabolism, to increase mitochondrial biogenesis or for weight loss.

A search of the literature reveals that there is no article suggesting the combination of liposomal encapsulated glutathione and arginine for the purpose of mitochondrial biogenesis and/or weight loss.

Management of Type 2 Diabetes

Management of type 2 diabetes generally managed by drugs in the categories known as sulfonylureas, metformin or Thiazolidinediones.

Thiazolidinediones such as, rosiglitazone and pioglitazone have become accepted medications for the treatment of type 2 diabetes, and both of these drugs work by increasing insulin sensitivity. It has been demonstrated that the mechanism of action of rosiglitazone and pioglitazone is centered on their ability to activate the peroxisome proliferator-activated receptor PPARγ, which is abundantly expressed in adipose tissue and is present in vasculature, colonic epithelium, and leukocytes (Wilson-Fritch). Normally fatty acids and eicosanoids bind to PPARγ, which activates the receptor causing it to migrate to the nucleus and DNA, activating a number of genes. It appears that PPARγ induces mitochondrial biogenesis in a way that increases fatty acid oxidation and markedly enhances oxygen consumption in these tissues and ultimately in the whole body energy metabolism with a resulting increase in insulin sensitivity (Wilson-Fritch). In spite of the biochemical prediction of benefit, research with pioglitazone teaches away from the expectation of weight loss as it was found that after 26 weeks of usage there was a dose dependent increase in body weight and BMI in the pioglitazone treated individuals of 2.0 to 4.5 Kgs⁸². The authors proposed that the PPARγ activation by pioglitazone alone activated the formation of more fat in the fat cells, particularly in subcutaneous fat cells. It is proposed that the use of the present invention will increase the efficacy of stimulation to the mitochondria biogenesis mechanism and improve the function of thiazolidinediones as well as insulin for the treatment of type 2 diabetes.

In addition to the actions describe above, pioglitazone, brand name “Actos” (made under license by Takeda Pharmaceuticals North America, Inc., and Eli Lilly Company of Indianapolis, Ind.), has been found to increase high-density lipoprotein (HDL). The present invention is proposed in combination with pioglitazone as a combination for raising HDL for the treatment of atherosclerosis. The preferred mode of the invention is the combination of pioglitazone 30 to 45 mg/day and Liposomal glutathione 800 mg (2 teaspoons), and l-arginine 1.0 to 2.5 gms twice a day.

Vitamin D₃ exerts a variety of functions in the body related to calcium homeostasis, cell proliferation and cell differentiation. Most of these actions are mediated through the control of target genes stimulated by the action of the vitamin D receptor (VDR). Binding to the vitamin D receptor results in a series of events leading to regulation of target genes and affects a wide variety of tissues including bone, kidney, cardiac and skeletal muscle⁸³. It has been demonstrated that PGC-1-α acts as a stimulator of the VDR and that both of these receptors are involved in developing skeletal muscle⁸³. The present invention is proposed in combination with vitamin D₃. The increased use of vitamin D is known to increase the number of vitamin D receptors and this will increase the rate of mitochondrial biogenesis progressing. The dose of vitamin D anticipated for function is in the range of 2000 to 50000 IU per day, with monitoring of the blood levels of Vit D (25OH) to be sure that there is both a response to the therapy and that the Vitamin D level does not go excessively high. The normal range of vitamin D in the blood is 20-100 ng/ml and a level of 50 to 75 ng/ml is the target level for good vitamin D function.

Low ATP levels are associated with the feeling of fatigue⁸⁴, individuals with chronic fatigue syndrome were observed to have a 20% reduction in oxidative metabolism and they were also noted to have decreased oxygen delivery to muscle after exercise.⁸⁵ The present invention is proposed for the treatment of chronic fatigue syndrome.

It has also been observed that the individuals with low ATP production experience more fatigue than individuals producing adequate ATP⁸⁶. Fatigue is related to mitochondrial abnormality⁸⁷ as well as decreased mitochondrial function⁸⁸ after oxidative stressors such as radiation or chemotherapy⁸⁹. The use of liposomal glutathione alone or in the form of the present invention is proposed to manage the oxidation stress increase mitochondrial biogenesis and increase the availability of ATP for management of the fatigue that accompanies decreased ATP production²⁷, from sources such as increased TNF-α, environmental toxins, and post radiation or chemotherapy for individuals who have undergone these therapies for cancer.⁹⁰. All the changes caused by ionizing radiation are compatible with mitochondrial failure, encompassing reduced production of ATP, generation of ROS, and accumulation of rhodamine 123 which reflect mitochondrial swelling or changes in the mitochondrial inner membrane⁹¹.

Chemotherapeutic agents used in treating various cancers have been demonstrated to increase oxidation stress of the proteins and lipids in the brain. The phenomenon is so common that it is referred to as “chemobrain”⁸⁹ and is characterized by forgetfulness, lack of concentration, dizziness and fatigue to the point of sleeping. It is proposed that either liposomal reduced glutathione alone or in the form of the present invention as a treatment for the symptoms of “chemobrain”. These symptoms are associated with decreased glutathione levels in brain tissue. The invention may be used between episodes of the administration of the chemotherapy agent or at the conclusion of the therapy. As the formation of ROS and Peroxynitrite occurs during radiation therapy⁹², strategies of mitigating or correcting damage to mitochondria have advantages in rehabilitating the individual and their tissues after radiation or chemotherapy will have advantages. It is proposed that liposomal glutathione alone or the present invention be provided to ameliorate the effects of radiation or chemotherapy.

Chemotherapy agents with which the present invention is intended include, but is not limited to:
Alkalating agents such as cisplatin, carboplatin, oxaliplatin, Busulfan, Cyclophoshamide and Melphalan
Antimetabolites such as azathioprine, mercaptopurine, pyrimdine, 5-Fluorouracil, Methotrexate and Fludarabine
Vinca alkaloids such as Vincristine, Vinblastine, Vinorelbine, Vindesine
Antitumor Antibiotics such as Bleomycin, Doxorubicin and Idarubicin
Mitotic Inhibitors including Taxanes such as pactitaxel, Docetaxel, Etoposide and Vinorelbine
Cyclophosphamide (Cytoxan, Neosar)

An embodiment of the present invention for use in individuals undergoing radionuclide exposure for either diagnostic purposes or as a therapy using radioactively tagged tumor specific modalities. These materials in general consist of a tumor targeting agent such as an antibody that targets tumor tissue to which a radioactive component has been attached. Liposomes tagged with radionuclide agents have been used for tumor imaging to stage cancers, image repeatedly and for the delivery of therapeutic doses of radionuclide such as technicium⁹⁹. Liposomes have been shown to be useful in carrying ^{99m}Tc to tumor targets. ^{99m}Tc is a preferred material for imaging compared to ¹¹¹In and ⁶⁷Ga based on aspects of availability, cost and better imaging characteristics. Specific characteristics of the liposome used in constructing a vehicle for the radionuclide could play a role in increasing the efficacy of the combination for both visualization and treatment of tumors. Because of the fragility of radiopharmaceuticals, a material that would easily and without disrupting the radiopharmaceutical would be a novel advance in their construction.

The combination of the radionuclide with a self forming liposome sold under the brand name “QuSome” by Biozone Laboratories, Inc. of Pittsburgh, California would be a real advantage. The Qusome self-forming liposome can be mixed with the intended radionuclide material at the time of its use, and literally “at the bedside”, prior to injection or ingestion if needed. Most liposomes use energy provided as heat, sonication, extrusion, or homogenization for their formation, which gives them a high energy state. Since every high-energy state tries to lower its free energy, many liposome formulations can experience problems with aggregation, fusion, sedimentation and leakage of liposome associated material. A thermodynamically stable liposome formulation which could avoid these problems is a technological advance in liposome construction. The additional advantage that the Qusome self-forming liposome is self forming at room temperature means that this is a true “mix and go” liposome that can be formed by mixing the lipid and an aqueous of lipid containing solution, without the worry that the contents will be altered, preserving the immunogenicity of the antigen and modulators. The resulting liposome is in a low free energy state so it remains stable and reproducible. This means that the QuSome self-forming liposome can be readily translated from bench top to large scale production without problem. The formulation of this embodiment is reviewed in example 4.

The QuSome self-forming liposome uses polyethyleneglycol (PEG) as a steric stabilizer and the resulting liposome is of a moderate size, 150 nm-250 nm. The combination of 150 nm-250 nm size and the PEG component is known to create long circulating liposomes. The size of the QuSome self-forming liposome allows them to be sterile filtered. These attributes allow the QuSome liposome encapsulating a radionuclide useful for targeting tumors with either diagnostic radionuclides or therapeutic radionuclides. The QuSome self-forming liposome is of such as size and the presence of the steric stability with PEG results in long circulation and an increased accumulation in the fine trabecular mesh of blood vessels supplying growing tumors. This characteristic will allow for improved diagnostics as more radionuclide accumulates around the tumor improving the image of scans. This characteristic of accumulating in the trabecular mesh of blood vessels leading to tumors will also leads to an improved therapeutic. The accumulation of QuSome self-forming liposomes in the blood vessel supply to tumors increases the radiation dosing to this area, creating damage to the tumor blood vessels creating an anti-angiogenic effect, resulting in a decreased supply of blood to the tumor and leading to death of tumor cells.

At the same time the present invention, liposomal glutathione in liposomes derived from lecithin that are more “fast acting” in terms or releasing their contents into the system can be administered to decrease the damage that radiation has on the surrounding tissues. It is proposed that the present invention be used to ameliorate the effects of chemotherapy and/or radiation that affect mitochondrial function resulting in tissue damage. This application applies to whether these exposures come from controlled exposures such as medical therapies or uncontrolled exposures as is seen with chemical toxicitics or radiation exposure from industrial, accidental or intentional situations such as poisonings or bombs.

### OBJECTIVES OF THE INVENTION

It is an objective of the invention to enable weight loss and reduce of oxidative stress and well as positively influencing mitochondrial biogenesis.

It is an objective of the invention to enable the prevention and treatment of insulin resistance and particularly insulin resistance in the elderly. Insulin resistance has been shown to occur in the elderly population associated with an increase in fat accumulation in muscle and liver and with a 40% decrease in mitochondrial oxidative phosphorylation (OXPHOS)⁹³. As these findings are consistent with an age related decline in mitochondrial function as previously discussed, the invention is useful in treating insulin resistance.

It is an objective of the invention to use vitamin D in addition to increase the number of receptor sites utilized by PGC-1-α in order to increase the stimulation for and the rate of mitochondrial biogenesis, which is an increase in the number and function of mitochondria⁶⁷.

It is an objective of the invention to be used for the treatment of chronic fatigue syndrome.

It is an objective of the invention to treat the fatigue that accompanies therapies utilizing chemotherapy or radiation for the treatment of various disease states such as cancer.

It is an objective of the invention to treat malaria and other intracellular diseases such as lyme disease.

Alternative biochemicals may be substituted for arginine in the formation of nitric oxide. The amino acid lysine has been demonstrated to form nitric oxide when added to the diet or supplemented in animal studies⁹⁴. Citrulline will also become incorporated in the pathways forming arginine and may be considered a substitute for arginine⁴¹ Agmatine is another substitute.

### PREFERRED MODE OF INVENTION

The combination of Liposomal glutathione 2500 mg per ounce with l-arginine 3000 mg per ounce designed to be ingested orally is the preferred mode of the present invention. The liposome for this mode is described in the Example numbers 1 and 2 uses the material derived from lecithin for the liposome.

While the preferred mode of the present invention is in the liposome composed of material derived from lecithin for oral use, a second preferred mode is the combination of liposomal reduced glutathione and l-arginine encapsulated in the Qusome for topical use for application to areas of excess fat. The Qusome is composed of fatty material that is readily absorbed through the skin and into fat tissue under the skin. The amount of materials is 2500 mg glutathione plus l-arginine 3000 mg per ounce in a cream for topical application. It is proposed that the topical Qusome encapsulating reduced glutathione and l-arginine will be used for topical application either alone or in conjunction with the oral ingestion of the present invention to increase the mitochondrial metabolism of cells such as adipocytes, which are fat cells. In addition, the combination of oral ingestion of the invention and the topical application of the invention in the Qusome may speed the resolution of fatty deposits in specific sites as well as an aid in wound healing by increasing local tissue mitochondrial biogenesis to support healing as well as increasing local blood flow by dilating the local vessels. Additional components of the topical may include forskolin, aminophyllin or yohimbe as supplemental materials to stimulate increased lypolysis of fat cells. Forskolin a labdane diterene that is produced from the plant Plectranthus barbatus and is known to raise levels of cyclic Adenosine Monophospate (cAMP). cAMP is a signal carrying molecule that is necessary for responses in cells. For example, cAMP is the signaling molecule that is triggered by nitric oxide that goes on to cause muscle relaxation. While important in regulating cell functions, too much cAMP in cells will cause problems such as the development of insulin resistance. Stimulants to cell metabolism such as catecholamines (epinephrine) or glutathione or an enzyme that prevents the breakdown of cAMP called cAMP-phosphodiesterase inhibitor, will increase cAMP and result in insulin resistance and slowing of fat metabolism in ⁹⁵. Aminophyllin is a methylxanthines, a group that also includes caffeine and theophylline, that is known to cause smooth muscle relaxation and also increase production of enzymes in cells and can inhibit macrophage inflammation and phagoeytosis⁹⁶. Additionally, it has been shown that responsiveness to insulin can be restored in fat cells by the beta l-adrenergic effect (bronchial dilation) of aminophyllin or the beta-antagonist, propranolol⁹⁵, which increases fat metabolism in cells. It is thought that the response to methyl xanthines occurs because of interaction with adenosine receptors, resulting in a restoration of their response to insulin⁹⁵. Yohimbe, an herb that is a natural alpha-2 antagonist, allowing increased beta adrenergic expression, also increases fat loss mechanisms and may be used as a component of either the oral or topical form of the invention.

The topical Qusome combination is manufactured as described in Example 4.

### ADDITIONAL APPLICATIONS

Additional applications that will benefit from the application of the present invention are the treatment of disease such as malaria, which is associated with both a decrease in arginine systemically and nitric oxide in the brain during acute malaria (Lopansri 2006). The present invention offers advantages that would not accompany the single administration of arginine to these individuals. As reviewed in the patent Guilford Patent Application II US 60/863,015; PCT\US06\60271 increasing the level of nitric oxide without providing liposomal encapsulated glutathione would not result in the formation of GSNO, which has been shown to be an inhibitor of a critical enzyme needed for the malaria parasite to infect red blood cells^{97,98}). The present invention is proposed as a method of directly or as an adjunct with chloroquine and aminoisoquinolines pharmacologics in the management and prevention and malaria. Additionally, the present invention is proposed in combination with a liposomal encapsulation of an extract of Artemisia, such as artesuate, which has been found useful in the management and prevention of malaria⁹⁹. The preferred mode of the combination for malaria in adults is Chloroquine 25 mg of salt/kg over 36-48 hours or 600 mg base (=1,000 mg salt) should be given initially, followed by 300 mg base (=500 mg salt) at 6, 24, and 48 hours after the initial dose for a total chloroquine dose of 1,500 mg base (=2,500 mg salt). Simultaneously liposomal glutathione 1200 mg+l-arginine 1000 mg is given every 4 hours for the first 48 hours and then every 6 hours in addition to Primaquine 15 mg once a day for fourteen days for 14 days.

An additional embodiment of the invention proposes the combination of Liposomal glutathione and l-arginine with colloidal silver. The preferred embodiment of this combination is Liposomal glutathione 1200 mg+arginine 1500 mg plus colloidal silver 32 ppm of silver nano particles 10 cc to be used three times a day for the treatment of acute malarial disease.

Individuals with chronic and active lyme disease often have fatigue accompanying their symptoms and have been shown to have decreased function of the enzyme glutathione peroxidase and increased markers of oxidative stress¹⁰⁰ and have also been demonstrated to have increased levels of TNF-α¹⁰¹. It is proposed that a similar combination of liposomal glutathione 1200 mg+arginine 1500 mg plus colloidal silver ppm of silver nano particles 10 cc to be used three times a day for the treatment of acute and chronic lyme disease.

The colloidal silver described in this embodiment may be obtained from American Biotech Laboratories of Alpine, Utah, USA.

A search of the literature reveals that there is no article suggesting the combination of liposomal encapsulated glutathione and arginine for the purpose of mitochondrial biogenesis and/or weight loss.

As used herein the term “agonist” or “agonist of eNOS or cNOS” refers to an agent that stimulates the bio-transformation of a substrate such as, for example, L-arginine to NO. An agonist of eNOS or cNOS includes, for example, an HMG-CoA reductase inhibitor. “HMG-CoA reductase (3-hydroxy-3-methylglutaryl-coenzyme A)” is the microsomal enzyme that catalyzes the rate limiting reaction in cholesterol biosynthesis. An “HMG-CoA reductase inhibitor” inhibits HMG-CoA reductase. HMG-CoA reductase inhibitors are also referred to as “statins.”

In another embodiment of the invention, the composition may further include a number of non-active compounds, such as effervescent combinations, diluents, buffers, preservatives, desiccants, thickeners, fillers, flavorings, sweeteners, colorings and any other excipients or non-active ingredients known in the art. The composition maybe in the form of a powder, liquid, capsule, tablet or chewing gum and/or may be formed as part of a food product. In a preferred embodiment, the composition is a powder that may be solubilized in a liquid for ingestion.

### OBJECTIVES OF THE INVENTION

The incidence of weight gain leading to obesity has developed to epidemic proportions in industrialized nations. While many theories have been proposed, this application proposes that there is a biochemical abnormality that can prevent weight loss it is an object of the present invention that it is a composition whose ingestion enables the function of the biochemistry for weight loss. At the same time, the composition of the present invention leads to an increase in the ability to feel satiety after eating, allowing the individual to avoid overeating. It is an object of the present invention to allow weight loss.

Insulin resistance has been shown to occur in the elderly population associated with an increase in fat accumulation in muscle and liver and with a 40% decrease in mitochondrial OXPHOS⁹³. As these findings are consistent with an age related decline in mitochondrial function as previously discussed, it is an objective of the invention to enable the treatment of insulin resistance and particularly insulin resistance in the elderly by increasing mitochondrial function and biogenesis.

It is an objective of the invention to be used as a combination to extend the function of mitochondria during aging and to delay the decline of mitochondrial function associated with aging.

It is an objective of the invention to be used for the treatment of chronic fatigue.

It is an objective of the invention to treat the fatigue that accompanies exposures to environmental toxins as well as therapies utilizing chemotherapy or radiation for the treatment of various disease states such as cancer.

It is an objective of the invention to use-vitamin D in addition to increase the number of receptor sites utilized by PGC-1-α in order to increase the stimulation for and the rate of mitochondrial biogenesis.

It is an objective of the invention to treat malaria and other intracellular diseases such as Lyme disease.

Alternative biochemicals may be substituted for arginine in the formation of nitric oxide. The amino acid lysine has been demonstrated to form nitric oxide when added to the diet or supplemented in animal studies⁹⁴. Citrultine will also become incorporated in the pathways forming arginine and may be considered a substitute for arginine

### Case Example 1

MR, a 60 year old woman, with diabetes requiring insulin therapy also has a long history of elevated blood pressure and increased weight. MR also has a long history of type 2 diabetes requiring insulin therapy on a twice daily basis. In spite of numerous attempts to lose weight the patient had been unable to lose weight and at the start of the usage of the present invention she was 5 feet 4.5 inches and weighed 230 pounds, which calculates to a Body Mass Index of 39.5.

### Case Example 2

AF, a 67 year old man who had a long history of elevated blood pressure and excess weight. AF has been on a weight control program for years. He has reduced his carbohydrates, balances carbohydrates, protein and fat. In spite of daily walks for exercise, he has not been able to lose weight. Two weeks before starting the present invention AF estimates his weight was “at least” 250 pounds. The individual is 5 feet, 9 inches tall and the BMI calculates to 37. At that time his blood pressure was significantly elevated at 210/110. He agreed to follow the advice of his physician regarding blood pressure medications. He also elected to start using liposomal glutathione. Two weeks later he elected to add l-arginine to the liposomal glutathione using doses of liposomal glutathione 800 mg morning and l-arginine 950 mg. with each of these ingested together twice a day. At week 5 his weight was documented at 239 pounds. At week 8 his weight was 228 pounds. 16 weeks after starting the present invention he relates his weight is 218 pounds, a BMI of 32. The loss of weight from the estimated level represents 32 pounds lost in 5 months and a reduction in BMI of 7 points. There is a documented reduction of 21 pounds over a 4 month period.

In addition to the weight loss, AF notes that he feels more relaxed and comfortable than he has in some time. He recounts feeling stressed and anxious on a continual basis in the past and since starting the present invention notes that his level of anxiety and irritability has decreased.

### DETAILED DESCRIPTION OF THE PREFERRED EMBODIMENTS

### Example 1

l-Arginine 1000 mg to 3000 mg is ingested orally followed
by the liposomal glutathione drink 420 mg per teaspoon,
which is constructed in the following manner.
Liposomal glutathione Drink or Spray 2500 mg per ounce

| Ingredient | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| Glutathione (reduced) | 8.25 |

Note:
Glutathione reduced 8.25 w/w % is 82.5 mg per ml.

A lipid mixture having components lecithin, and glycerin were commingled in a large volume flask and set aside for compounding.

In a separate beaker, a water mixture having water, glycerin, glutathione were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400×magnification confirmed presence of both multilamellar lipid vesicles (MLV) and unilamellar lipid vesicles.

The preferred embodiment includes the variations of the amount of glutathione to create less concentrated amounts of glutatbione. The methods of manufacture described in Keller et al, U.S. Pat. No. 5,891,465, Apr. 6, 1999, are incorporated into this description. The preferred liposomal glutathione is available from Your Energy Systems, Inc. of Palo Alto, Calif.

### Example 2

Liposomal glutathione Drink or Spray 2500 mg
per ounce with l-arginine 3000 mg per ounce.

| Ingredient | % w/w |
|---|---|
| Deionized Water | 64.4 |
| Glycerin | 15.00 |
| Lecithin | 1.50 |
| Potassium Sorbate | 0.10 |
| (optional spoilage retardant) | |
| Glutathione (reduced) | 8.25 |
| L-arginine | 10.0 |

A lipid mixture having components lecithin, and glycerin were commingled in a large volume flask and set aside for compounding.

In a separate beaker, a water mixture having water, glycerin, glutathione were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic Stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture described in Keller et al, U.S. Pat. No. 5,891,465, Apr. 6, 1999, are incorporated into this description.

### Example 3

Glutathione + l-arginine liposomal capsule Formulation

| Ingredient | Concentration % |
|---|---|
| Sorbitan oleate | 2.0 |
| Glutathione (reduced) | 45.0 |
| l-arginine | 45.0 |
| Deionized water | 4.0 |
| Potassium sorbate | 0.2 |
| Polysorbate 20 | 2.0 |
| Phospholipon 90 (DPPC) | 2.0 |

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. The resulting one gram capsule contains 450 mg reduced glutathione and 450 mg l-arginine. Large scale manufacturing methods for filling gel caps, such as the rotary die process, are the preferred method for commercial applications. The liposomal glutathione for this invention is and was made by Biozone Laboratories, Inc. of Pittsburgh, California and sold by Your Energy Systems, Inc. of Palo Alto, Calif.

### Example 4

Embodiment number three of the present invention includes the creation of liposome suspension using a self-forming, thermodynamically stable liposomes formed upon the adding of a diacylglycerol-PEG lipid to an aqueous solution when the lipid has appropriate packing parameters and the adding occurs above the melting temperature of the lipid. The method described by Keller et al, U.S. Pat. No. 6,610,322 is incorporated into this description.

Most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.

The present embodiment teaches liposome suspensions which are thermodynamically stable at the temperature of formation. The formulation of such suspensions is achieved by employing a composition of lipids having several fundamental properties. First, the lipid composition must have packing parameters which allow the formation of liposomes. Second, as part of the head group, the lipid should include polyethyleneglycol (PEG) or any polymer of similar properties which sterically stabilizes the liposomes in suspension. Third, the lipid must have a melting temperature which allows it to be in liquid form when mixed with an aqueous solution.

By employing lipid compositions having the desired fundamental properties, little or no energy need be added when mixing the lipid and an aqueous solution to form liposomes. When mixed with water, the lipid molecules disperse and self assemble as the system settles into its natural low free energy state. Depending on the lipids used, the lowest free energy state may include small unilamellar vesicle (SUV) liposomes, multilamellar vesicle (MLV) liposomes, or a combination of SUVs and MLVs.

In one aspect, the invention includes a method of preparing liposomes. The method comprises providing an aqueous solution; providing a lipid solution, where the solution has a packing parameter measurement of Pₐ (Pₐ references the surface packing parameter) between about 0.84 and 0.88, a Pᵥ (Pᵥ references the volume packing parameter) between about 0.88 and 0.93, (See, D.D. Lasic, Liposomes, From Physics to Applications, Elsevier, p. 51 1993), and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleoylglycerol-PEG-12, either alone or as one of the lipids in a mixture. The method may further comprise providing an active compound, in this case glutathione (reduced) and combining the active compound with the lipid solution and the aqueous solution. In the situation where the self forming liposome (“QuSome” by Biozone Laboratories, Inc. of Pittsburgh, California, is used to create a radiopharmaceutical, the radionuclide will first be created with the ligand selected to target a particular tissue. The does would be that for the desired radiopharmaceutical as would be known to a reasonably skilled practitioner. Thereafter, the radiopharmaceutical be used as the active substance. The active substance radiopharmaceutical would be combined with the self-forming lipid solution and any desired the aqueous solution. The selected dose would be selected by a dosimeter, and administered. Because the liposomes will pass into the digestive tract, the dose may be given orally, but also intravenously, or for certain types of cancers, by injection.

Additional variations of accomplishing this embodiment are described in Keller et al U.S. Pat. No. 6,610,322.

The accumulation of QuSome self-forming liposomes in the blood vessel supply to tumors increases the radiation dosing to this area, creating damage to the tumor blood vessels creating an anti-angiogenic effect as well, resulting in a decreased supply of blood to the tumor and leading to death of tumor cells. By using the QuSome self-forming liposomes, and the liposomal glutathione alone, or liposomal glutathione and arginine, the tumor is selectively preferred as the target at the same time as normal cells are better protected.

The above process, apparatus and resulting composition related to use is adaptable to the stabilization and preservation of virtually all radionuclides whatever the solvent used for initial composition. Some preferred applications include stabilization of radiolabeled peptides, [18 F] deoxyglucose, radiolabelled annexin, 99 mTc-annexin, radiolabelled monocyte chemoattractant protein. i.e. 125-I-(MCP-1), radiolabelled Dopamine transporter agents, (S)-N-(1-ethylpyrrolidin-2-ylmethyl)-2-hydro-xy-3-iodo-6-methoxybenzamide (3-IBZM) (More generally “BZM,), (S)-N-(1-ethylpyrrolidin-2-ylmethyl)-2-hydroxy-5-iodo-6-methoxybenzamide (5-IBZM), 1-123-2-betacarbomethoxy-3-beta(4-iodophenyl) N-(3-fluoro propyl) nortropane (“CIT” or “beta-CIT”) and various tropane derivatives, 1-123 fatty acids, particularly for cardiovascular imaging, radiolabelled octreotide or radiolabelled depreotide, HEDP (diagnostic skeletal imaging or treatment of metastatic bone pain), radiolabelled antibodies, both polyclonal and monoclonal, with selective affinities for tumor-associated antigens diagnosis or in situ radiotherapy of malignant tumors such as melanomas), and ligands with selective affinity for the hepatobiliary system (the liver-kidney system), including 2,6-dimethylacetanilideiminodi-acetic acid and the family of other imidoacetic acid group-containing analogs thereof (collectively referred to herein as “HIDA agents”), mono-, di- and polyphosphoric acids and their pharmaceutically-acceptable salts including polyphosphates, pyrophosphates, phosphonates, diphosphonates and imidophosphonates. Preferred ligands are 1-hydroxyethylidene diphosphonate, methylene diphosphonate, (dimethylamino)methyl diphosphonate, methanehydroxydiphosphonate, and imidodiphosphonate (for bone-scanning and alleviation of pain); strontium 89 ethylene diamine tetramethylene phosphate, samarium 153-ethylene diamine tetramethylene phosphate, radiolabelled monoclonal antibodies, 99m-Tc HMPAO (hexamethylproplyene amine oxime), yttrium 90-labeled ibritumomab tiuxetan (Zevalin.RTM. Registered Trademark of Biogen Idcc, Inc.), and meta-iodo-benzyl guanidine. Ethylene diamine tetramethylene phosphate and ethylene diamine tetramethylene phosphoric acid and the pharmaceutically related mono-, di- and polyphosphoric acids and their pharmaceutically-acceptable salts including polyphosphates, pyrophosphates, phosphonates, diphosphonates and imidophosphonates are collectively called EDTMP.

Suitable radionuclides which are well-known to those skilled in the art include radioisotopes of copper, technetium-99m, rhenium-186, rhenium-188, antimony-127, lutetium-177, lanthanum-140, samarium-153, radioisotopes of iodine, indium-111, gallium-67 and -68, chromium-51, strontium-89, radon-222, radium-224, actinium-225, californium-246 and bismuth-210. Other suitable radionuclides include F-18, C-11, Y-90, Co-55, Zn-62, Fe-52, Br-77, Sr-89, Zr-89, Sm-153, Ho-166, and TI-201.

RECOMMENDED USE in conjunction with radiation therapy or chemotherapy in the dose of radiopharmaceutical selected by a person reasonably skilled in the art is:

(1 ounce is 5.56 teaspoons.)
1 teaspoon of oral liposomal glutathione reduced+l-arginine contains approximately 440 mg GSH+500 mg L-arginine.

Suggested dose depends on body weight. Recommended amounts are for daily use.

### Adult Dosing

Recommended dose for adult is two teaspoons twice a day for a 70 Kg person.

For adults of 100 Kg the dose is 2 teaspoons three times a day.

For adults of 150 Kg the dose is 2 teaspoons four times a day.

### Children's Dosing

DETERMINE DAILY DOSE BY BODY WEIGHT: for use twice a day.

Under 30 lbs: ¼ teaspoon=110 mg GSH+125 mg l-arginine
30-60 lbs: ½ teaspoon=220 mg GSH+250 mg l-arginine
60-90 lbs: ¾ teaspoon=330 mg GSH+375 mg l-arginine
90-120 lbs: 1 teaspoon=440 mg GSH+500 mg l-arginine
120-150 lbs: 1½ teaspoon=660 mg GSH+750 mg l-arginine
Over 150 lbs: 2 teaspoons=880 mg GSH+1000 mg l-arginine
Gently stir liposomal glutathione into the liquid of your choice.
No refrigeration is required after opening.

Also, if a stabilized and lyophilized radiopharmaceutical that is reconstituted at on-site at administration according to the art of Wolfangel, U.S. Pat. No. 5,219,556, Jun. 15, 1993, or Kuperus, U.S. Publ. 20050281737, Dec. 22, 2005 is created, or other art involving a lyophilized radiopharmaceutical, the invention proposes utilizing a self-forming liposome in solution, reconstituting the radiopharmaceutical with the solution with the self-forming liposome, and administering the radiopharmaceutical, now in the self-forming liposome, to the patient. Liposomal glutathione may be added to the solution prior to administration.

### Example 5

### Diabetes Management

The present invention is proposed in combination with pioglitazone as a combination for raising HDL for the treatment of atherosclerosis. The preferred mode of the invention is the combination of pioglitazone 30 to 45 mg/day and Liposomal glutathione 800 mg (2 teaspoons), and 1-arginine 1.0 to 2.5 gms twice a day.

Management of Malaria

The present invention is proposed as a method of directly or as an adjunct with chloroquine and aminoisoquinolines pharmacologics in the management and prevention and malaria. Additionally, the present invention is proposed in combination with a liposomal encapsulation of an extract of Artemisia, such as artesuate, which has been found useful in the management and prevention of malaria⁹⁹. The preferred mode of the combination for malaria in adults is Chloroquine 25 mg of salt/kg over 36-48 hours or 600 mg base (=1,000 mg salt) should be given initially, followed by 300 mg base (=500 mg salt) at 6, 24, and 48 hours after the initial dose for a total chloroquine dose of 1,500 mg base (=2,500 mg salt). Simultaneously liposomal glutathione 1200 mg+l-arginine 1000 mg is given every 4 hours for the first 48 hours and then every 6 hours in addition to Primaquine 15 mg once a day for fourteen days for 14 days.

An additional embodiment of the invention proposes the combination of Liposomal glutathione and l-arginine with colloidal silver. The preferred embodiment of this combination is Liposomal glutathione 800 mg+arginine 1500 mg plus colloidal silver 32 ppm of silver nano particles 10 cc to be used three times a day for the treatment of acute malarial disease.

Lyme Disease

Individuals with chronic and active lyme disease often have fatigue accompanying their symptoms and have been shown to have decreased function of the enzyme glutathione peroxidase and increased markers of oxidative stress¹⁰⁰ and have also been demonstrated to have increased levels of TNF-α¹⁰¹. It is proposed that a similar combination of liposomal glutathione 800 mg+arginine 1500 mg plus colloidal silver 32 ppm of silver nano particles 10 cc to be used three times a day for the treatment of acute and chronic lyme disease.

The colloidal silver described in this embodiment may be obtained from American Biotech Laboratories of Alpine, Utah, USA.

The invention is not meant to be limited to the disclosures, including best mode of invention herein, and contemplates all equivalents to the invention and similar embodiments to the invention for humans, mammals and plant science. Equivalents include combinations with or without stabilizing agents and adjuncts that assist in reservation, and their pharmacologically active racemic mixtures, diastereomers and enantiomers and their pharmacologically acceptable salts in combination with suitable pharmaceutical carriers.
1. Kelm M, Schrader J. Control of coronary vascular tone by nitric oxide. Circulation research. 1990; 66(6): 1561-1575.
2. Cooke J P. Asymmetrical Dimethylarginine: The Uber Marker? Circulation. 2004; 109(15):1813-1818.
3. Aggarwal B B, Shishodia S, Ashikawa K, Bharti A C. The role of TNF and its family members in inflammation and cancer: lessons from gene deletion. Current drug targets. 2002; 1(4):327-341.
4. Warne J P. Tumour necrosis factor alpha: a key regulator of adipose tissue mass. The Journal of endocrinology. 2003; 177(3):351-355.
5. Sethi J K, Hotamisligil G S. The role of TNF alpha in adipocyte metabolism. Seminars in cell & developmental biology. 1999; 10(1): 19-29.
6. Skoog T, Dichtl W, Boquist S, Skoglund-Andersson C, Karpe F, Tang R, Bond M G, de Faire U, Nilsson J, Eriksson P, Hamsten A. Plasma tumour necrosis factor-alpha and early carotid atherosclerosis in healthy middle-aged men. European heart journal. 2002; 23(5):376-383.
7. Hotamisligil G S, Shargill N S, Spiegelman B M. Adipose expression of tumor necrosis factor-alpha: direct role in obesity-linked insulin resistance. Science. 1993; 259(5091):87-91.
8. Hotamisligil G S, Arner P, Caro J F, Atkinson R L, Spiegelman B M. Increased adipose tissue expression of tumor necrosis factor-alpha in human obesity and insulin resistance. The Journal of clinical investigation. 1995; 95(5):2409-2415.
9. Valerio A, Cardile A, Cozzi V, Bracale R, Tedesco L, Pisconti A, Palomba L, Cantoni O, Clementi E, Moncada S, Carruba M O, Nisoli E. TNF-alpha downregulates eNOS expression and mitochondrial biogenesis in fat and muscle of obese rodents. The Journal of clinical investigation. 2006; 116(10):2791-2798.
10. Sohal R S, Svensson I, Brunk U T. Hydrogen peroxide production by liver mitochondria in different species. Mechanisms of ageing and development. 1990; 53(3):209-215.
11. Xu H, Barnes G T, Yang Q, Tan G, Yang D, Chou C J, Sole J, Nichols A, Ross J S, Tartaglia L A, Chen H. Chronic inflammation in fat plays a crucial role in the development of obesity-related insulin resistance. The Journal of clinical investigation. 2003; 112(12):1821-1830.
12, Weisberg S P, McCann D, Desai M, Rosenbaum M, Leibel R L, Ferrante A W, Jr. Obesity is associated with macrophage accumulation in adipose tissue. The Journal of clinical investigation. 2003; 112(12):1796-1808.
13. Cinti S, Mitchell G, Barbatelli G, Murano I, Ceresi E, Faloia E, Wang S, Fortier M, Greenberg A S, Obin M S. Adipocyte death defines macrophage localization and function in adipose tissue of obese mice and humans. Journal of lipid research. 2005; 46(11):2347-2355.
14. Bojunga J, Dresar-Mayert B, Usadel K H, Kusterer K, Zeuzem S. Antioxidative treatment reverses imbalances of nitric oxide synthase isoform expression and attenuates tissue-cGMP activation in diabetic rats. Biochemical and biophysical research communications. 2004; 316(3):771-780.
15. Munoz-Fernandez M A, Fresno M. The role of tumour necrosis factor, interleukin 6, interferon-gamma and inducible nitric oxide synthase in the development and pathology of the nervous system. Progress in neurobiology. 1998; 56(3):307-340.
16. Neels J G, Pandey M, Hotamisligil G S, Samad F. Autoamplification of tumor necrosis factor-alpha: a potential mechanism for the maintenance of elevated tumor necrosis factor-alpha in male but not female obese mice. The American journal of pathology. 2006; 168(2):435-444.
17. Visser M, Bouter L M, McQuillan G M, Wener M H, Harris T B. Elevated C-reactive protein levels in overweight and obese adults. Jama. 1999; 282(22):2131-2135.
18. de Rekeneire N, Peila R, Ding J, Colbert L H, Visser M, Shorr R I, Kritchevsky S B, Kuller L H, Strotmeyer E S, Schwartz A V, Vellas B, Harris T B. Diabetes, hyperglycemia, and inflammation in older individuals: the health, aging and body composition study. Diabetes care. 2006; 29(8):1902-1908.
19. Belyaeva E A, Korotkov S M. Mechanism of primary Cd2+-induced rat liver mitochondria dysfunction: discrete modes of Cd2+ action on calcium and thiol-dependent domains. Toxicology and applied pharmacology. 2003; 192(1):56-68.
20. Cheng Y J, Liu M Y. Modulation of tumor necrosis factor-alpha and oxidative stress through protein kinase C and P42/44 mitogen-activated protein kinase in lead increases lipopolysaccharide-induced liver damage in rats. Shock (Augusta, Ga. 2005; 24(2):188-193.
21. Ishii Y, Partridge C A, Del Vecchio P J, Malik A B. Tumor necrosis factor-alpha-mediated decrease in glutathione increases the sensitivity of pulmonary vascular endothelial cells to H2O2. The Journal of clinical investigation. 1992; 89(3):794-802.
22. Cadenas E, Davies K J. Mitochondrial free radical generation, oxidative stress, and aging. Free radical biology & medicine. 2000; 29(3-4):222-230.
23. Frisard M, Ravussin E. Energy metabolism and oxidative stress: impact on the metabolic syndrome and the aging process. Endocrine. 2006; 29(1):27-32.
24. Fernandez-Checa J C, Garcia-Ruiz C, Colell A, Morales A, Mari M, Miranda M, Ardite E. Oxidative stress: role of mitochondria and protection by glutathione. BioFactors (Oxford, England). 1998; 8(1-2):7-11.
25. Steffen M, Sarkela T M, Gybina A A, Steele T W, Trasseth N J, Kuehl D, Giulivi C. Metabolism of S-nitrosoglutathione in intact mitochondria. The Biochemical journal 2001; 356(Pt 2):395402.
26. Cleeter M W, Cooper J M, Darley-Usmar V M, Moncada S, Schapira A H. Reversible inhibition of cytochrome c oxidase, the terminal enzyme of the mitochondrial respiratory chain, by nitric oxide. Implications for neurodegenerative diseases. FEBS Lett. 1994; 345(1):50-54.
27. Wlodek D, Gonzales M. Decreased energy levels can cause and sustain obesity. Journal of theoretical biology. 2003; 225(1):33-44.
28. Brown G C, Cooper C E. Nanomolar concentrations of nitric oxide reversibly inhibit synaptosomal respiration by competing with oxygen at cytochrome oxidase. FEBS Lett. 1994; 356(2-3):295-298.
29. Wilson-Fritch L, Nicoloro S, Chouinard M, Lazar M A, Chui P C, Leszyk J, Straubhaar J, Czech M P, Corvera S. Mitochondrial remodeling in adipose tissue associated with obesity and treatment with rosiglitazone. The Journal of clinical investigation. 2004; 114(9): 1281-1289.
30. Wardle J, Brodersen N H, Cole T J, Jarvis M J, Boniface D R. Development of adiposity in adolescence: five year longitudinal study of an ethnically and socioeconomically diverse sample of young people, in Britain. BMJ (Clinical research ed. 2006; 332(7550):1130-1135.
31. Haslam D W, James W P. Obesity. Lancet. 2005; 366(9492):1197-1209.
32. King H, Aubert R E, Herman W H. Global burden of diabetes, 1995-2025: prevalence, numerical estimates, and projections. Diabetes care. 1998; 21(9):1414-1431.
33. Huse D M, Oster G, Killen A R, Lacey M J, Colditz G A. The economic costs of non-insulin-dependent diabetes mellitus. Jama. 1989; 262(19):2708-2713.
34. Menshikova E V, Ritov V B, Fairfull L, Ferrell R E, Kelley D E, Goodpaster B H. Effects of exercise on mitochondrial content and function in aging human skeletal muscle. The journals of gerontology. 2006; 61(6):534-540.
35. Patti M E, Butte A J, Crunkhom S, Cusi K, Berria R, Kashyap S, Miyazaki Y, Kohane I, Costello M, Saccone R, Landaker E J, Goldfine A B, Mun E, DeFronzo R, Finlayson J, Kahn C R, Mandarino L J. Coordinated reduction of genes of oxidative metabolism in humans with insulin resistance and diabetes: Potential role of PGCI and NRFI. Proceedings of the National Academy of Sciences of the United States of America. 2003; 100(14):8466-8471.
36. Kern P A, Saghizadeh M, Ong J M, Bosch R J, Deem R, Simsolo R B. The expression of tumor necrosis factor in human adipose tissue. Regulation by obesity, weight loss, and relationship to lipoprotein lipase. The Journal of clinical investigation. 1995; 95(5):2111-2119.
37. Ritov V B, Menshikova E V, He J, Ferrell R E, Goodpaster B H, Kelley D E. Deficiency of subsarcolemmal mitochondria in obesity and type 2 diabetes. Diabetes. 2005; 54(1):8-14.
38. Stump C S, Short K R, Bigelow M L, Schimke J M, Nair K S. Effect of insulin on human skeletal muscle mitochondrial ATP production, protein synthesis, and mRNA transcripts. Proceedings of the A National Academy of Sciences of the United States of America. 2003; 100(13):7996-8001.
39. Peterson J, Kanai A J, Pearce L L. A mitochondrial role for catabolism of nitric oxide in cardiomyocytes not involving oxymyoglobin. Am J Physiol Heart Circ Physiol. 2004; 286(1):H55-58.
40. Short K R, Vittone J L, Bigelow M L, Proctor D N, Nair K S. Age and aerobic exercise training effects on whole body and muscle protein metabolism. Am j Physiol Endocrinol Metab. 2004; 286(1):E92-101.
41. Wu G, Morris S M, Jr. Arginine metabolism: nitric oxide and beyond. The Biochemical journal. 1998; 336 (Pt 1):1-17.
42. Cicek E, Yildiz M, Delibas N, Bahceti S. The effects of thyroid scintigraphy studies on oxidative damage in patients. Acta physiologica Hungarica. 2006; 93(2-3):131-136.
43. Arai M, Imai H, Koumura T, Yoshida M, Emoto K, Umeda M, Chiba N, Nakagawa Y. Mitochondrial phospholipid hydroperoxide glutathione peroxidase plays a major role in preventing oxidative injury to cells. J Biol Chem. 1999; 274(8):4924-4933.
44. Mason M G, Nicholls P, Wilson M T, Cooper C E. Nitric oxide inhibition of respiration involves both competitive (heme) and noncompetitive (copper) binding to cytochromc c oxidase. Proceedings of the National Academy of Sciences of the United States of America. 2006; 103(3):708-713.
45. Brown G C, Borutaite V. Nitric oxide, cytochrome c and mitochondria. Biochemical Society symposium. 1999; 66:17-25.
46. Clementi E, Brown G C, Foxwell N, Moncada S. On the mechanism by which vascular endothelial cells regulate their oxygen consumption. Vol 96; 1999:1559-1562.
47. Clementi E, Brown G C, Feelisch M, Moncada S. Persistent inhibition of cell respiration by nitric oxide: Crucial role of S-nitrosylation of mitochondrial complex I and protective action of glutathione. PNAS. 1998; 95(13):7631-7636.
48. Clementi E, Brown G C, Foxwell N, Moncada S. On the mechanism by which vascular endothelial cells regulate their oxygen consumption. PNAS. 1999; 96(4):1559-1562.
49. Nakamura M, Thourani V H, Ronson R S, Velez D A, Ma X L, Katzmark S, Robinson J, Schmarkey L S, Zhao Z Q, Wang N P, Guyton R A, Vinten-Johansen J. Glutathione reverses endothelial damage from peroxynitrite, the byproduct of nitric oxide degradation, in crystalloid cardioplegia. Circulation. 2000; 102(19 Suppl 3):III332-338.
50. Wu M, Pritchard K A, Jr., Kaminski P M, Fayngersh R P, Hintze T H, Wolin M S. Involvement of nitric oxide and nitrosothiols in relaxation of pulmonary arteries to peroxynitrite. The American journal of physiology. 1994; 266(5 Pt 2):H2108-2113.
51. Giulivi C, Poderoso J J, Boveris A. Production of Nitric Oxide by Mitochondria. J. Biol. Chem. 1998; 273(18):11038-11043.
52. Ghafourifar P, Asbury M L, Joshi S S, Kincaid E D. Determination of mitochondrial nitric oxide synthase activity. Methods in enzymology. 2005; 396:424-444.
53. Cadenas E. Mitochondrial free radical production and cell signaling. Molecular aspects of medicine. 2004; 25(1-2):17-26.
54. Cai J, Jones D P. Superoxide in Apoptosis. MITOCHONDRIAL GENERATION TRIGGERED BY CYTOCHROME c LOSS. J. Biol. Chem. 1998; 273(19):11401-11404.
55. Buckley B J, Whorton A R. Adaptive responses to peroxynitrite: increased glutathione levels and cystine uptake in vascular cells. American journal of physiology. 2000; 279(4):C1168-1176.
56. Brito P M, Mariano A, Almeida L M, Dinis T C. Resveratrol affords protection against peroxynitrite-mediated endothelial cell death: A role for intracellular glutathione. Chemico-biological interactions. 2006; 164(3):157-166.
57. Knight T R, Ho Y S, Farhood A, Jaeschke H. Peroxynitrite is a critical mediator of acetaminophen hepatotoxicity in murine livers: protection by glutathione. The Journal of pharmacology and experimental therapeutics. 2002; 303(2):468-475.
58. Fan Q, Yang X C, Cao X B, Wang S Y, Yang S L, Liu X L, Gao F. Glutathione reverses peroxynitrite-mediated deleterious effects of nitroglycerin on ischemic rat hearts. Journal of cardiovascular pharmacology. 2006; 47(3):405-412.
59. Beckman J S, Koppenol W H. Nitric oxide, superoxide, and peroxynitrite: the good, the bad, and ugly. The American journal of physiology. 1996; 271(5 Pt 1):C1424-1437.
60. Zweier J L, Fertmann J, Wei G. Nitric oxide and peroxynitrite in postischemic myocardium. Antioxidants & redox signaling. 2001; 3(1):11-22.
61. Shen L, Sevanian A. OxLDL induces macrophage gamma-GCS-HS protein expression: a role for oxLDL-associated lipid hydroperoxide in GSH synthesis. Journal of lipid research. 2001; 42(5):813-823.
62. Sikorski J A. Oral cholesteryl ester transfer protein (CETP) inhibitors: a potential new approach for treating coronary artery disease. Journal of medicinal chemistry. 2006; 49(1):1-22.
63. Aspnes L E, Lee C M, Weindruch R, Chung S S, Roecker E B, Aiken J M. Caloric restriction reduces fiber loss and mitochondrial abnormalities in aged rat muscle. Vol 11; 1997:573-581.
64. Civitarese A E, Carling S, Heilbronn L K, Hulver M H, Ukropcova B, Deutsch W A, Smith S R, Ravussin E. Calorie Restriction Increases Muscle Mitochondrial Biogenesis in Healthy Humans. PLoS Med. 2007; 4(3):e76.
65. Ingram D K, Young J, Mattison J A. Calorie restriction in nonhuman primates: assessing effects on brain and behavioral aging. Neuroscience. 2007.
66. Nisoli E, Tonello C, Cardile A, Cozzi V, Bracale R, Tedesco L, Falcone S, Valerio A, Cantoni O, Clementi E, Moncada S, Carruba M O. Calorie Restriction Promotes Mitochondrial Biogenesis by Inducing the Expression of eNOS. Science. 2005; 310(5746):314-317.
67. Nisoli E, Carruba M O. Nitric oxide and mitochondrial biogenesis. Journal of cell science. 2006; 119(14):2855-2862.
68. Lin S-J, Ford E, Haigis M, Liszt G, Guarente L. Calorie restriction extends yeast life span by lowering the level of NADH. Genes Dev. 2004; 18(1):12-16.
69. Hickner R C, Kemeny G, Stallings H W, Manning S M, McIver K L. Relationship between body composition and skeletal muscle eNOS. International journal of obesity (2005). 2006; 30(2):308-312.
70. Pouleur H. Diastolic dysfunction and myocardial energetics. European heart journal. 1990; 11 Suppl C:30-34.
71. Simoneau J-A, Veerkamp J H, Turcotte L P, Kelley D E. Markers of capacity to utilize fatty acids in human skeletal muscle: relation to insulin resistance and obesity and effects of weight loss. FASEB J. 1999; 13(14):2051-2060.
72. Reis D J, Regunathan S. Agmatine: an endogenous ligand at imidazoline receptors is a novel neurotransmitter. Annals of the New York Academy of sciences. 1999; 881:65-80.
73. Li G, Regunathan S, Barrow C J, Eshraghi J, Cooper R, Reis D J. Agmatine: an endogenous clonidine-displacing substance in the brain. Science. 1994; 263(5149):966-969.
74. Sastre M, Galea E, Feinstein D, Reis D J, Regunathan S. Metabolism of agmatine in macrophages: modulation by lipopolysaccharide and inhibitory cytokines. The Biochemical journal. 1998; 330 (Pt 3):1405-1409.
75. Ishikawa T, Misonou T, Ikeno M, Sato K, Sakamaki T. N omegahydroxyagmatine: a novel substance causing endothelium-dependent vasorelaxation. Biochemical and biophysical research communications. 1995; 214(1):145-151.
76. Reis D J, Regunathan S. Is agmatine a novel neurotransmitter in brain? Trends in pharmacological sciences. 2000; 21(5): 187-193.
77. Salvi M, Battaglia V, Mancon M, Colombatto S, Cravanzola C, Calheiros R, Marques M P, Grillo M A, Toninello A. Agmatine is transported into liver mitochondria by a specific electrophoretic mechanism. The Biochemical journal. 2006; 396(2):337-345.
78. Reis D J, Yang X C, Milner T A. Agmatine containing axon terminals in rat hippocampus form synapses on pyramidal cells. Neuroscience letters. 1998; 250(3):185-188.
79. Yang X C, Reis D J. Agmatine selectively blocks the N-methyl-D-aspartate subclass of glutamate receptor channels in rat hippocampal neurons. The Journal of pharmacology and experimental therapeutics. 1999; 288(2):544-549.
80. Regunathan S. Agmatine: biological role and therapeutic potentials in morphine analgesia and dependence. The AAPS journal. 2006; 8(3):E479-484.
81. Uzbay I T, Yesilyurt O, Celik T, Ergun H, Isimer A. Effects of agmatine on ethanol withdrawal syndrome in rats. Behavioural brain research. 2000; 107(1-2):153-159.
82. Miyazaki Y, Matsuda M, DeFronzo R A. Dose-Response Effect of Pioglitazone on Insulin Sensitivity and Insulin Secretion in Type 2 Diabetes. Diabetes care. 2002; 25(3):517-523.
83. Savkur R S, Bramlett K S, Stayrook K R, Nagpal S, Burris T P. Coactivation of the human vitamin D receptor by the peroxisome proliferator-activated receptor gamma coactivator-1 alpha. Molecular pharmacology. 2005; 68(2):511-517.
84. Park J H, Phothimat P, Oates C T, Hernanz-Schulman M, Olsen N J. Use of P-31 magnetic resonance spectroscopy to detect metabolic abnormalities in muscles of patients with fibromyalgia. Arthritis and rheumatism. 1998; 41(3):406-413.
85. McCully K K, Natelson B H. Impaired oxygen delivery to muscle in chronic fatigue syndrome. Clin Sci (Lond). 1999; 97(5):603-608; discussion 611-603.
86. Pouw E M, Schols A M, van der Vusse G J, Wouters E F. Elevated inosine monophosphate levels in resting muscle of patients with stable chronic obstructive pulmonary disease. American journal of respiratory and critical care medicine. 1998; 157(2):453-457.
87. Fattal O, Budur K, Vaughan A J, Franco K. Review of the literature on major mental disorders in adult patients with mitochondrial diseases. Psychosomatics. 2006; 47(1):1-7.
88. Nicolson G L. Lipid replacement/antioxidant therapy as an adjunct supplement to reduce the adverse effects of cancer therapy and restore mitochondrial function. Pathol Oncol Res. 2005; 11(3):139-144.
89. Joshi G, Sultana R, Tangpong J, Cole M P, St Clair D K, Vore M, Estus S, Butterfield D A. Free radical mediated oxidative stress and toxic side effects in brain induced by the anti cancer drug adriamycin: insight into chemobrain. Free radical research. 2005; 39(11):1147-1154.
90. Dagnelie P, Pijls-Johannesma M, Lambin P, Beijer S, De Ruysscher D, Kempen G. Impact of fatigue on overall quality of life in lung and breast cancer patients selected for high-dose radiotherapy. Ann Oncol. 2007.
91. Lee J H, Kim S Y, Kil I S, Park J-W. Regulation of ionizing radiation-induced apoptosis by mitochondrial NADP+-dependent isocitrate dehydrogenase. J. Biol. Chem. 2007:M700303200.
92. Kanai A, Zabbarova 1, Amoscato A, Epperly M, Xiao J, Wipf P. Mitochondrial targeting of radioprotectants using peptidyl conjugates. Organic & biomolecular chemistry. 2007; 5(2):307-309.
93. Petersen K F, Befroy D, Dufour S, Dziura J, Ariyan C, Rothman D L, DiPietro L, Cline G W, Shulman G I. Mitochondrial dysfunction in the elderly: possible role in insulin resistance. Science. 2003; 300(5622):1140-1142.
94. Liaudet L, Gnaegi A, Rosselet A, Markert M, Boulat O, Perret C, Feihl F. Effect of L-lysine on nitric oxide overproduction in endotoxic shock. British journal of pharmacology. 1997; 122(4):742-748.
95. Elias D, Rapoport M, Cohen I R, Shechter Y. Desensitization of the insulin receptor by antireceptor antibodies in vivo is blocked by treatment of mice with beta-adrenergic agonists. The Journal of clinical investigation. 1988; 81(6):1979-1985.
96. Di Donato A, Draetta G F, Illiano G, Tufano M A, Sommese L, Galdiero F. Do porins inhibit the macrophage phagocyting activity by stimulating the adenylate cyclase? Journal of cyclic nucleotide and protein phosphorylation research. 1986; 11(2):87-97.
97. Sharma A, Eapen A, Subbarao S K. Parasite Killing in Plasmodium vivax Malaria by Nitric Oxide: Implication of Aspartic Protease Inhibition. Vol 136; 2004:329-334.
98. Sharma A, Eapen A, Subbarao S K. Parasite Killing in Plasmodium vivax Malaria by Nitric Oxide: Implication of Aspartic Protease Inhibition. Journal of biochemistry. 2004; 136(3):329-334.
99. Yeka A, Banek K, Bakyaita N, Staedke S G, Kamya M R, Talisuna A, Kironde F, Nsobya S L, Kilian A, Slater M, Reingold A, Rosenthal P J, Wabwire-Mangen F, Dorsey G. Artemisinin versus nonartemisinin combination therapy for uncomplicated malaria: randomized clinical trials from four sites in Uganda. PLoS Med. 2005; 2(7):e190.
100. Pancewicz S A, Herrnanowska-Szpakowicz T, Makarewicz-Plonska M, Witek A, Farbiszewski R, Zajkowska J, Michalska B. [Decreased antioxidant-defence mechanisms in cerebrospinal fluid (CSF) in patients with tick-borne encephalitis (TBE)]. Neurologia i neurochirurgia polska. 2002; 36(4):767-776.
101. Kisand K E, Prukk T, Kisand K V, Luus S M, Kalbe I, Uibo R. Propensity to excessive proinflammatory response in chronic Lyme borreliosis. Apmis. 2007; 115(2):134-141.

What is claimed is:

1. A composition for enabling weight loss or appetite suppression in a mammalian patient comprising:
reduced glutathione in a liposomal formulation capable of administration intravenously, orally, dermally or mucosally; and
l-arginine.

2. The composition according to claim 1, further comprising:
a compound selected from the group of materials that increase nitric oxide production, including agmatine, and citrulline.

3. The pharmaceutical composition according to claim 1, further comprising:
said l-arginine being contained in said liposomal formulation.

4. A composition for treating infection in a mammalian patient comprising:
reduced glutathione in a liposomal formulation capable of administration intravenously, orally, dermally or mucosally; and
l-arginine;
and colloidal silver.

5. The composition according to claim 4, further comprising:
said l-arginine being contained in said liposomal formulation.

6. The composition according to claim 5, further comprising:
said colloidal silver being contained in said liposomal formulation.

7. The composition according to claims 5, 6 or 7, further comprising:
said infection being lyme disease.

8. The composition according to claims 5, 6, or 7, further comprising:
said infection being lyme disease; and
a therapeutic dose of selenium.

9. The composition according to claims 5, 6 or 7, further comprising:
said infection being malaria.

10. The composition according to claims 5, 6, or 7, further comprising:
said infection being lyme disease; and
a therapeutic dose of selenium.

11. A composition in combination with a cholesterol-ester transfer protein (“CETP”) inhibitor for ameliorating the negative effects of CETP inhibitors comprising:
reduced glutathione in a liposomal formulation capable of administration orally, dermally or mucosally; and
a CETP inhibitor.

12. The composition according to claim 11, further comprising:
l-arginine.

13. The composition according to claim 11, further comprising:
said l-arginine being contained in said liposomal formulation.

14. The composition according to claim 12, further comprising:
said CETP inhibitor being contained in said liposomal formulation.

15. The composition according to claims 11, 12, 13 or 14, further comprising:
said CETP inhibitor being torcetrapib.

16. The composition according to claims 11, 12, 13 or 14, further comprising:
said CETP inhibitor being torcetrapib; and
a therapeutic dose of selenium.

17. A composition for treatment of vascular disease and diabetes in a mammalian patient, comprising:
reduced glutathione in a liposomal formulation capable of administration orally, dermally or mucosally; and
a therapeutic dose of l-arginine; and
a therapeutic dose of a thiazolidinedione.

18. The pharmaceutical composition according to claim 17, further comprising: at least one of said therapeutic dose of l-arginine and said therapeutic dose of thiazolidinedione being contained in said liposomal formulation.

19. The composition according to claims 1 through 6, or claims 11 through 14, or claims 17 and 18, further comprising:
a therapeutic dose of selenium.

20. A method of enhancing mitochondrial biogenesis comprising:
administering reduced glutathione in a liposomal formulation capable of administration intravenously, orally, dermally or mucosally; and
administering l-arginine.

21. The method according to claim 20, further comprising:
said l-arginine being contained in said liposomal formulation.

22. A method of ameliorating the negative effects of a cholesterol-ester transfer protein (“CETP”) inhibitor while administering said CETP inhibitor comprising:
administering reduced glutathione in a liposomal formulation capable of administration orally, dermally or mucosally; and
administering a CETP inhibitor.

23. The method according to claim 22, further comprising:
administering l-arginine.

24. The method according to claim 23, further comprising:
at least one of said l-arginine and said CETP inhibitor being contained in said liposomal formulation.

25. A method of enabling and facilitating weight loss comprising:
administering reduced glutathione in a liposomal formulation capable of administration intravenously, orally, dermally or mucosally; and
administering l-arginine.

26. The method according to claim 25, further comprising:
said l-arginine being contained in said liposomal formulation.

27. The method according to claims 20, 21, 22, 23, 24, 25 or 26, further comprising:
a therapeutic dose of selenium.
